(19) United States
(12) Patent Application Publication
(10) Pub. No.: US 2005/0119289 A1
(43) Pub. Date: Jun. 2, 2005

(54) Enhancing treatment of MDR cancer with adenosine A3 antagonists

(76) Inventors: Pier Andrea Borea, Ferrara (IT); Pier Giovanni Baraldi, Ferrara (IT); Shih-Fong Chen, Apex, NC (US); Edward Leung, Cary, NC (US)

Correspondence Address: EDWARDS & ANGELL, LLP, P.O. BOX 55874, BOSTON, MA, 02205, US

(21) Appl. No.: 10/600,116

(22) Filed: Jun. 20, 2003

Related U.S. Application Data
(60) Provisional application No. 60/391,009, filed on Jun. 24, 2002.

(51) Int. Cl. A61K 31/70; A61K 31/655; A61K 31/522

(52) U.S. Cl. 514/267; 514/283; 514/263.32; 514/23; 514/151

(57) ABSTRACT

The present invention discloses the use of high affinity adenosine A₃ receptor antagonists for enhancing chemotherapeutic treatment of cancers expressing adenosine A₃ receptors and cancers expressing P-glycoprotein or MRP. In preferred embodiments, adenosine A₃ receptor antagonists are administered before or during administration of a taxane family, vinca alkaloid, camptothecin or antibiotic chemotherapeutic agent.

### BACKGROUND OF THE INVENTION

The present invention relates to medicaments useful in the treatment of cancer used in combination with cytotoxic agents. Surprisingly, it has been found that adenosine A₃ antagonists synergistically enhance cytotoxic treatment and counter some forms of multi-drug resistance.

Adenosine has been linked to tumor development. Increased adenosine concentration has been reported inside tumoral masses. It has been speculated that it represents the anti-tumor agent that prevents tumor growth in muscle tissue in vivo and that impairs malignant cell growth and survival in vitro. However, it is known that adenosine acts as cyto-protective agent during ischemic damage in brain and heart. Adenosine is known to be released in hypoxia. Numerous studies have shown adenosine to protect cells in the heart from ischemic damage.

Adenosine has been shown to have protective roles in numerous animal models and in man (Am. J. Cardiol. 79(12A):44-48 (1997). For example, in the heart, both the A₁ and A₃ receptors offer protection against ischemia (Am. J. Physiol., 273(42)H501-505 (1997). However, it is the A₃ receptor that offers sustained protection against ischemia (PNAS 95:6995-6999 (1998). The ability of adenosine to protect tumor cells against hypoxia has not been recognized by others.

Adenosine interacts with cell surface receptors that are glycoproteins coupled to different members of G protein family. By now four adenosine receptors have been cloned and characterised: A₁, A_{2A}, A_{2B} and A₃. Selective antagonists for the A₃ receptor have been proposed for use as anti-inflammatory and antiischemic agents in the brain. Recently, A₃ antagonists have been under development as antiasthmatic, antidepressant, anti-arrhythmic, renal protective, antiparkinson and cognitive enhancing drugs. For example, U.S. Pat. No. 5,646,156 to Marlene Jacobson et al. inhibits eosinophil activation by using selected A₃ antagonists.

Recent studies in myocytes have shown the adenosine A₃ receptors to be responsible for long-term protection against ischemia (Liang and Jacobson, PNAS, 95:6995-6999 (1998)). While the present inventors have hypothesized that adenosine plays a protective role in other cell types, including tumor cells, in addition to myocytes, no efforts have been made to limit the protective effect of adenosine on tumor cells.

Recently, the inventors have demonstrated that adenosine promotes contradictory effects on cell survival and proliferation by simultaneous stimulation of different adenosine receptors. In particular, A₃ adenosine receptor impairs cell proliferation but also improves cell survival. Furthermore, it has been demonstrated that A₃ receptor blocks UV irradiation-induced apoptosis in mast-like cells (RBL-2H3).

U.S. Pat. No. 6,326,390 to Leung et al. also identifies the use of adenosine A₃ receptor in anti-neoplastic treatment. Leung discloses the use of adenosine A₃ antagonists for treatment of tumors containing elevated levels of the A₃ receptors. U.S. Pat. No. 6,326,390 is incorporated by reference.

Inventors have verified the concentration of adenosine A₃ receptors in human cancer cells that is elevated in the cancerous tumors compared to non-cancerous tumors or normal tissue. Table 1 and FIG. 1 indicate elevated levels of adenosine A₃ receptors found by the inventors in human A375 (human melanoma), Panc-1 (human pancreatic carcinoma), MX-1 (human breast carcinoma), PC-1 (human prostate carcinoma), HT29 (human colon carcinoma), and SKMES (human lung carcinoma).

TABLE 1
Abundance of A₃ receptors in human solid tumors and
melanoma. Data shown are equilibrium binding parameters
at 4° C. expressed as dissociation constant, K_{D} (nM),
and B_{MAX} (fmol/mg protein) for [³H]MRE 3008F20
derived from saturation experiments to human A₃
adenosine receptors expressed in tumour tissues.

| Tumor types | K_{D} | B_{MAX} | FIG. |
|---|---|---|---|
| Human malignant | 1.9 ± 0.2 | 256 ± 31 | 1A |
| m lanoma A375 | | | |
| Human c lon DLD1 | 2.1 ± 0.3 | 434 ± 40 | 1B |
| Human pancreatic | 2.6 ± 0.3 | 249 ± 34 | 1C |
| MiaPaCa | | | |
| Human pancreatic | 2.7 ± 0.1 | 441 ± 46 | 1D |
| Panc1 | | | |
| Human breast tumor | 1.9 ± 0.2 | 435 ± 50 | 1E |
| MX1 | | | |
| Human lung squamour | 2.4 ± 0.6 | 169 ± 20 | 1F |
| cancinoma SKMES | | | |
| Human pancreatic PC3 | 3.1 ± 0.1 | 379 ± 40 | 1G |
| Human colon HT29 | 3.3 ± 0.3 | 213 ± 23 | 1H |

U.S. Pat. No. 6,066,642 to Jacobson et al. discloses the expression of the apoptosis-inducing protein bak in response to the adenosine A₃ agonist CI-IB-MECA at 10 μM. The response was suppressed by adenosine A₃ antagonists in two cell lines tested. Although not conclusive, when combined with the recent results of the inventors, it is a reasonable hypothesis that adenosine A₃ receptors are present in the Jacobson tested cancer cells having induced bak. These include human HL-60 leukemia, MCF7 breast adenocarcinoma, U-937 histiocytic lymphoma, and 132 iN astrocytoma cells. However, not all cancers show CL-IB-MECA induced expression of bak. For example, U373 astrocytoma cells studied by Jacobson did not show bak expression. U.S. Pat. No. 6,066,642 is incorporated by reference.

In the current chemotherapeutic treatment of human cancer, side effects associated with the chemotherapeutic agent are often severe. The use of paclitaxel or docetaxel (both taxane family medicaments) for the treatment of neoplastic diseases is limited by acute hypersensitivity reactions experienced in many patients. For example, docetaxel administered at 100 mg/m² causes acute hypersensitivity reaction in 13% of patients, and severe hypersensitivity reaction in 1.2%. Due to these reactions, patients are normally premedicated with oral corticosteroids.

Similarly, side effects associated with the use of vinca alkaloids often limit the useful dosages. For example, vincristine has been reported to be dose limited due to neurotoxicity. An enhancing agent providing a neuro-protective effect is therefore desirable.

Chemotherapeutic agents are also costly to produce and provide to patients. If the agents can be used at reduced dosages, both the cost and the extent of undesirable side effects can be similarly reduced.

Another often-encountered challenge with chemotherapeutic treatment is limitations in effectiveness due to the cancerous growth or cells developing multidrug-resistance (MDR). As most cancer cells are genetically unstable they are prone to mutations likely to produce drug resistant cells.

Multi-drug resistance is the name given to the circumstance when a disease does not respond to a treatment drug or drugs. MDR can be either intrinsic, which means the disease has never been responsive to the drug or drugs, or it can be acquired, which means the disease ceases responding to a drug or drugs that the disease had previously been responsive to. MDR is characterized by cross-resistance of a disease to more than one functionally and/or structurally unrelated drugs. MDR in the field of cancer, is discussed in greater detail in “Detoxification Mechanisms and Tumor Cell Resistance to Anticancer Drugs,” by Kuzmich and Tew, particularly section VII “The Multidrug-Resistant Phenotype (MDR),” Medical Research Reviews, Vol. 11, No. 2, 185-217, (Section VII is at pp. 208-213) (1991); and in “Multidrug Resistance and Chemosensitization: Therapeutic Implications for Cancer Chemotherapy,” by Georges, Sharom and Ling, Advances in Pharmacology, Vol. 21, 185-220 (1990).

Different MDR mechanisms have been reported. One form of multi-drug resistance (MDR) is mediated by a membrane bound 170-180 kD energy-dependent efflux pump designated as pleitotropic-glycoprotein or P-glycoprotein (P-gp) that is codified by MDR-1 gene (Endicott JA, Annu Rev Biochem 1989). P-glycoprotein has been shown to play a major role in the intrinsic and acquired resistance of a number of human tumors. Drugs that act as substrates for and are consequently detoxified by P-gp include the vinca alkaloids (vincristine and vinblastine), anthracyclines (Adriamycin), and epipodophyllotoxins (etoposide).

Recently, MDR-1 gene has been identified as an additional risk factor in advanced ovarian cancer. In the study by D. S. Alberts et al, patients with phase III ovarian cancer were screened for MDR-1. Ovarian cancer patients with high levels of MDR-1 survived an average of 9.8 months. The patients having low or no MDR-1 expression survived an average of 30 months or more.

While P-gp associated MDR is a major factor in tumor cell resistance to chemotherapeutic agents, it is clear that the phenomenon of MDR is multifactorial and involves a number of different mechanisms. One such alternative pathway for resistance to anthracyclines involves the emergence of a 190 kD protein (p190) that is not P-gp. See, T. McGrath, et al., Biochemical Pharmacolology, 38:3611 (1989). The protein p190 is not found exclusively on the plasma membrane but rather appears to be localized predominantly in the endoplasmic reticulum. See, e.g., D. Marquardt, and M. S. Center, Cancer Research, 52:3157 (1992).

The work of Cole and Deeley isolated a single open reading frame of 1531 amino acids encoding a protein designated as multidrug resistance-associated protein (MRP). As reported by Fan et al., the MRP protein is thought to be the same as the 190 kD protein. MRP has been observed in breast cancer, human leukemia, small cell lung cancer, human large cell lung cancer, fibrosarcoma, adenocarcinoma, thyroid cancer, and cervical cancer. Chen reports that MRP is associated with multi-drug resistance to camptothecin and its analogs.

Other MDR has been reported that is neither P-gp nor MRP related. (Kellen editor, Alternative Mechanims of Multidrug Resistance in Cancer, Birkhauser, 1995). The results obtained by inventors are consistent for using adenosine A₃ antagonists for countering P-gp or MRP multi-drug resistance, but not other forms of MDR.

α-[3-[[2-(3,4-Dimethoxyphenyl)ethyl]methylamino]propyl]-3,4-dimethoxy-α-(1-methylethyl)benzeneacetonitrile (Verapamil) has been utilized as a medicament to counter the effect of P-gp associated MDR. Verapamil blocks L-type calcium channels and is used as a potent vasodilator of coronary and peripheral vessels and decreases myocardial oxygen consumption. Due to the Verapamil physiological effects, MDR use of Verapamil must be limited to patients not having low blood pressure, congestive heart failure, sinoatrial (SA) or atrioventricular (AV) node conduction disturbances, digitalis toxicity, Wolff-Parkinson-White syndrome, and further not being medicated with beta-blockers or Quinidine.

Recognizing that P-gp is also adenosine-5′-triphosphate (ATP) dependent, another proposed method of countering MDR is to inhibit ATP synthesis in the cancerous cells. U.S. Pat. No. 6,210,917 to Carson et al. discloses the use of L-alanosine and other adenosine kinase inhibitors for countering MDR. In addition U.S. Pat. No. 6,391,884 identifies ATP-depleting agents including 2-deoxyglucose, cyanine, oligomycin, valinomycin and azide, as well as salts and derivatives thereof. Approaches relying upon ATP depletion or inhibition in countering MDR have yet to receive clinical success. However, it is reasonably expected that combining ATP depleting agents with the adenosine A₃ antagonists of the present invention will work to advantage. U.S. Pat. Nos. 6,210,917 and 6,391,884 are incorporated herein.

As a result, it is seen that there is a need for chemotherapeutic agent enhancers and compounds that prevent or counter MDR either alone or with ATP depleting agents in cancer treatment but without the limitations imposed by Verapamil.

The use of a combination of therapeutic agents is common in the treatment of neoplastic diseases. For example, paclitaxel (Taxol®) has been approved by the U.S. FDA for use with cisplatin in the treatment of ovarian carcinoma. U.S. Pat. No. 5,908,835 to Bissery et al. Claims synergy of using paclitaxel or docetaxel in combination with an anthracycline antibiotic such as daunorubicin or doxorubicin. Similarly, U.S. Pat. No. 5,728,687 to Bisser et al. Claims synergy of using paclitaxel or docetaxel in combination with an alkylating agent, epidophyllotoxin, antimetabolite, or vinca alkaloid. However, such combinations heretofore have not included the use of adenosine receptor antagonists and in particular A₃ receptor antagonists.

U.S. Pat. No. 5,646,156 to Marlene Jacobson et al. discloses the use of adenosine A₃ receptor antagonists to inhibit eosinophil activation and degranulation and thereby prevent such conditions as asthma and hypersensitivity.

It is therefore an object of the present invention to provide compositions and methods of enhancing the treatment of neoplastic cells by minimizing or eliminating the protective effect of adenosine on cells with the use of adenosine A₃ receptor antagonists. It is a further object of the present invention to provide compositions and methods suitable for countering P-gp and/or MRP associated multi-drug resistance.

It is further an object of the present invention to provide compositions and methods that reduce taxane induced hypersensitivity in patients.

### BRIEF SUMMARY OF THE INVENTION

The present invention discloses the use of high affinity adenosine A₃ receptor antagonists for enhancing chemotherapeutic treatment of cancers expressing adenosine A₃ receptors and countering multi-drug resistance in cancers expressing P-glycoprotein or MRP. In preferred embodiments, adenosine A₃ receptor antagonists are administered before or during administration of a taxane family, vinca alkaloid, camptothecin or antibiotic compound. Preferred high affinity A₃ receptor antagonists include compounds of the following formulas wherein the substituents are as defined herein:

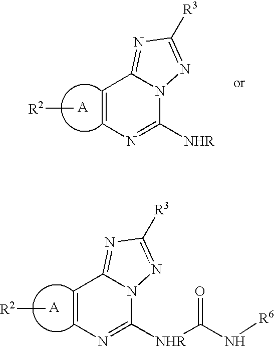

### BRIEF DESCRIPTION OF THE DRAWINGS

FIGS. 1A through 1H illustrate the saturation of [³H]-MRE 3008-F20 binding to A₃ adenosine receptors in human cancers. K_{D} and Bₘₐₓ values are reported in Table 1. Values are the means and S.E. of the mean of three separate experiments performed in triplicate. In the inset the Scatchard plot of the same data is shown.

FIG. 2 illustrates colony formation assay of A375 cells. Cells were treated with different drugs and colonies were counted after 7 days. The values represent the mean±SEM of four independent experiments. D=DMSO (control); T=paclitaxel, 0.75 ng/ml; M=MRE 3008F20, 10 μM; C═CI-IB-MECA, 10 μM; TM=paclitaxel plus MRE 3008F20; TC=paclitaxel plus CI-IB-MECA treated cells. *P<0.01 TM versus T. Analysis was by ANOVA followed by Dunnett's test.;

FIG. 3A through 3D illustrate typical dose response curves of A375 cells exposed to increasing concentrations of cytotoxic agents vindesine (FIG. 3A, FIG. 3B) and Taxol™ brand paclitaxel (FIG. 3C, FIG. 3D). The curves with open symbols represent the cytotoxic agent alone. The curves with closed symbols represent the cytotoxic agent in the presence of 10 μM MRE 3008F2010. FIG. 3A and FIG. 3C illustrate G₂/M phases arrest calculated as percentage of untreated cells (control). FIG. 3B and FIG. 3D illustrate the accumulation (percentage of total living cells) of the sub-G₁ (apoptosis) population. Cells were fixed in 70% ethanol, stained with PI, and analysed by flow cytometry.;

FIG. 4A through 4F illustrate induction of G₂/M phases arrest by MRE 3008F20 on exponentially growing A375 cells treated with paclitaxel or with vindesine. A375 cells are treated for 24 hours with drug-vehicle as control (FIG. 4A, FIG. 4D); with 1 nM vindesine (FIG. 4B), with 25 ng/ml paclitaxel (FIG. 4E); with 1 nM vindesine plus 10 μM MRE 3008F20 (FIG. 4C); or with 25 ng/ml paclitaxel plus 10 μM MRE 3008F20 (FIG. 4F). Cells were fixed in 70% ethanol, stained with PI, and analysed by flow cytometry. The percentage of cells at G₁, S and G₂/M phases was quantified. Apoptotic cells (Apo) were also detected.;

FIG. 5A illustrates dose-response curve for G₂/M phases arrest of A375 cells exposed to increasing concentrations of A₃ adenosine receptor antagonists in the presence of 1 nM vindesine. Cells were fixed in 70% ethanol, stained with PI, and analysed by flow cytometry.;

FIG. 5B illustrates comparison between enhancing activity to vindesine by adenosine receptor antagonists (SEC₅₀) and binding affinity to A₃ adenosine receptors (Kⱼ) in A375 cells (r=0.96; P<0.01).

FIG. 6A through 6D illustrate flow chromatogram for Rhodamine 123 (Rh 123) accumulation by A375 cells and HeL023 cells. FIG. 6A illustrates the accumulation by A375 cells. FIG. 6B illustrates the accumulation by A375 cells in the presence of 10 μM MRE 3008F20. FIG. 6C illustrates the accumulation by HeL023 cells. FIG. 6D illustrates the accumulation by HeL023 cells in the presence of 10 μM MRE 3008F20. In all cases, F_{MAX} represents the maximum load of Rh 123 (gray filled area). F_{RES} shows residual Rh 123 fluorescence after the P-gp mediated drug efflux was allowed for 3 hours (black filled area). Rh 123 unstained cell chromatogram is reported as unfilled area.

FIG. 7A illustrates the effect of inhibitors of MEK-(PD98059), ERK 1/2-(U0126) and p38^{MAPK}-(SB203580)-signalling on the G₂/M phases arrest induced by paclitaxel (filled bars) and by vindesine (empty bars). The residual G₂/M phases arrest is reported as the percentage of control cells (cells treated with paclitaxel plus MRE 3008F20 for TU, TS, TP or with vindesine plus MRE 3008F20, for VU, VS, VP.;

FIG. 7B illustrates the effect of inhibitors of MEK-(PD98059), ERK 1/2-(U0126) and p38MAPK-(SB203580)-signalling on apoptosis induced by paclitaxel. T=paclitaxel (25 ng/ml), V=vindesine 1 nM, U=U0126 30 μM, P═PD98059 20 μM, S═SB203580 1 μM, M=MRE 3008F20 10 μM, C=cells treated with metabolic inhibitor vehicle (DMSO) (control). Each bar represents the mean±SE of four independent experiments performed on A375 cells. P<0.01 as follows: *TU versus internal control (paclitaxel plus MRE 3008F20); #VU versus internal control (vindesine plus MRE 3008F20); §P<0.05: 2 versus 1, 4 versus 3, 6 versus 5 and 8 versus 7. Analysis was by ANOVA followed by Dunnett's test.

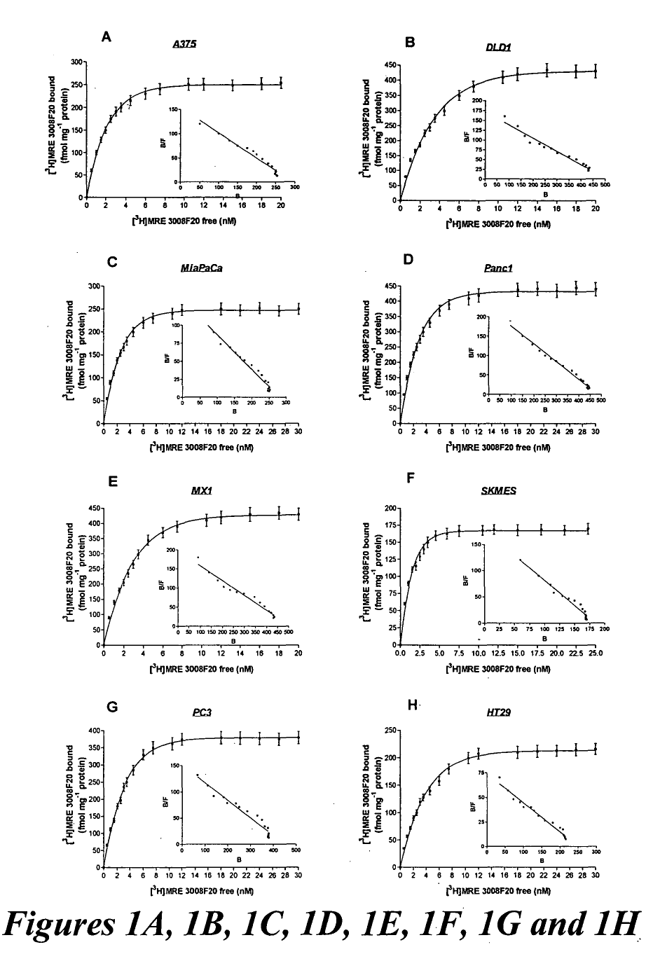

### DETAILED DESCRIPTION OF THE INVENTION

Inventors have discovered that high affinity adenosine A₃ receptor antagonists are useful as enhancers for many chemotherapeutic treatment of adenosine A₃ receptor expressing cancers. Surprisingly, high affinity adenosine A₃ receptor antagonists also counter P-glycoprotein (P-gp) effuse pump multi-drug resistance (MDR). Finally, high affinity adenosine A₃ receptor antagonists are helpful in reducing or ameliorating taxane induced hypersensitivity.

As used herein, “a high affinity adenosine A₃ receptor antagonist” refers to compounds that prevent the decrease in intracellular cAMP caused by activation of the A₃ adenosine receptor by adenosine agonists (for example CI-IB-MECA) and have measured affinity binding of less than 50 nM. Preferable high affinity adenosine A₃ receptor antagonists include compounds of the following formula and pharmaceutical salts thereof: 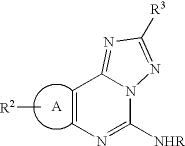
wherein:
A is imidazole, pyrazole, or triazole;
R is —C(X)R¹, —C(X)—N(R¹)₂, —C(X)OR¹, —C(X)SR¹, —SOₙR¹, —SOₙSR¹, or —SOₙ—N(R¹)₂;
R¹ is hydrogen, alkyl, substituted alkyl, alkenyl, substituted alkenyl, alkynyl, substituted alkynyl, aryl, substituted aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms;
R² is hydrogen, alkyl, substituted alkyl, alkenyl, aralkyl, substituted aralkyl, heteroaryl, substituted heteroaryl or aryl;
R³ is furan, pyrrole, thiophene, benzofuran, benzypyrrole, benzothiophene, optionally substituted with one or more substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aryl, substituted aryl, aryloxy, azido, carboxyl, carboxylalkyl, cyano, halo, nitro, heteroaryl, heteroaryloxy, heterocyclic, heterocyclooxy, aminoacyloxy, thioalkoxy, substituted thioalkoxy, —SO-alkyl, —SO-substituted alkyl, —SO-aryl, —SO-heteroaryl, —SO₂-alkyl, —SO₂-substituted alkyl, —SO2-aryl, —SO2-heteroaryl, and trihalomethyl;
X is O, S, or NR¹.

Even more preferable, as high affinity adenosine A3 receptor antagonist are “phenyl-carbamoyl-amino” compounds of the following formula and pharmaceutical salts thereof: 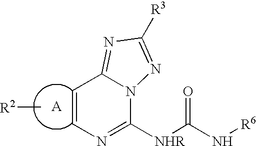
wherein:
A is imidazole, pyrazole, or triazole;
R² is hydrogen, alkyl, substituted alkyl, alkenyl, aralkyl, substituted aralkyl, heteroaryl, substituted heteroaryl or aryl;
R³ is furan; and
R⁶ is aryl, substituted aryl, heteroaryl, substituted heteroaryl, heterocycle or substituted heterocycle.

Chemotherapeutic Agents—The high affinity adenosine A₃ receptor antagonists can be administered alone or in combination with other chemotherapeutic cancer agents. Chemotherapeutic cancer agents are defined as agents that attack and kill cancer cells.

Chemotherapeutic cancer agents include numerous compounds such as taxane compounds and derivatives. Although taxane compounds were initially extracted from the Pacific yew tree, Taxus brevifolia. They include, for example, paclitaxel and its derivatives or docetaxel and its derivatives. Additional taxane derivatives and methods of synthesis are disclosed in U.S. Pat. No. 6,191,287 to Holton et al., U.S. Pat. No. 5,705,508 to Ojima et al., U.S. Pat. Nos. 5,688,977 and 5,750,737 to Sisti et. al., U.S. Pat. No. 5,248,796 to Chen et al., U.S. Pat. No. 6,020,507 to Gibson et al., U.S. Pat. No. 5,908,835 to Bissery, all of which are incorporated by reference.

Some chemotherapeutic cancer agents are mitotic inhibitors (vinca alkaloids). These include vincristine, vinblastine, vindesine and Navelbine™ (vinorelbine,5′-noranhydroblastine).

Similarly, chemotherapeutic cancer agents include topoisomerase I inhibitors such as camptothecin compounds. As used herein, “camptothecin compounds” include Camptosar™ (irinotecan HCL), Hycamtin™ (topotecan HCL) and other compounds derived from camptothecin and its analogues.

Another category of chemotherapeutic cancer agents are podophyllotoxin derivatives such as etoposide, teniposide and mitopodozide.

Other chemotherapeutic cancer agents are alkylating agents, which alkylate the genetic material in tumor cells. These include cisplatin, cyclophosphamide, nitrogen mustard, trimethylene thiophosphoramide, carmustine, busulfan, chlorambucil, belustine, uracil mustard, chlornaphazin, and dacarbazine.

Additional chemotherapeutic cancer agents are antimetabolites for tumor cells. Examples of these types of agents include cytosine arabinoside, fluorouracil, methotrexate, mercaptopurine, azathioprime, and procarbazine.

An additional category of chemotherapeutic cancer agents includes antibiotics. Examples include doxorubicin, bleomycin, dactinomycin, daunorubicin, mithramycin, mitomycin, mytomycin C, and daunomycin. There are numerous liposomal formulations commercially available for these compounds.

Also, other chemotherapeutic cancer agents include anti-tumor antibodies, dacarbazine, azacytidine, amsacrine, melphalan, ifosfamide and mitoxantrone.

As used herein, the term “A₃ expressing cancers” refers to human cancers that express the adenosine A₃ receptor or that otherwise comprise elevated concentrations of adenosine A₃ receptors. Elevated concentration is determined by comparison to normal, non-cancerous tissues of a similar cell type. Eamples of A₃ expressing cancers include, without limitation, human leukemia, melanoma, pancreatic carcinoma, breast carcinoma, prostrate carcinoma, colon carcinoma, lung carcinomamalignant melanomas, histiocytic lymphoma, and some forms of astrocytoma cells.

As used herein, “enhancement” refers to a synergistic effect as determined from measurement of the enhanced factor, as defined below. In general, an enhanced factor of two or greater is considered synergistic while an enhanced factor greater than one may be synergistic. For example, if one of the compounds has little individual chemotherapeutic effect, an enhanced factor greater than one indicates a synergistic effect is occurring.

As used herein, “adenosine kinase inhibitors” refers to compounds identified as adenosine kinase inhibitors in U.S. Pat. No. 6,210,917 to Carson et al and such other compounds as have comparable effect in depleting the target cells of adenosine 5′-triphosphate.

In a preferred embodiment of treating A₃ expressing cancers, a high affinity adenosine A₃ receptor antagonist and a chemotherapeutic cancer agent are administered to the patient. The combination therapy enhances the effect of the chemotherapeutic cancer agent and prevents multi-drug resistance from developing. As is shown below, the present invention is not effective for enhancing all forms of chemotherapeutic cancer agents. The chemotherapeutic cancer agents showing desirable response to the present invention are typical of agents noted for developing P-gp or MRP class multi-drug resistance. Examples include, without limitation, taxane compounds, vinca alkaloids, camptothecins and antibiotics useful as chemotherapeutic agents.

Similarly, the combination therapy can be used for treating cancers that have already developed multi-drug resistance. In this case, the high affinity adenosine A₃ receptor antagonist counters the existing MDR while further enhancing the effect of the chemotherapeutic cancer agent. As is shown below, the present invention is not effective for all forms of MDR cancers. The MDR cancers showing desirable response to the present invention are in the P-gp and MRP classes.

When the chemotherapeutic cancer agents is a compound of the taxane family, the high affinity adenosine A₃ receptor antagonist is preferably administered either before or during administration of the taxane compound. This is done to reduce the incidence of hypersensitivity to the taxane agent. Commercially available compounds of the taxane family are paclitaxel (commercially available under the tradename TAXOL from Bristol-Myers Squibb Company, Princeton, N.Y. and as a generic drug from IVAX Corp, Miami, Fla.) and docetaxel (commercially available under the tradename TAXOTERE from Aventis Pharmaceuticals, Collegeville, Pa.) It is noted that additional taxane family chemotherapeutic cancer agents are presently in development and testing.

The compounds can be administered in a time-release manner when permitted by the chemotherapeutic agent. Suitable time-release devices are well known to those of skill in the art. For example, the time-release effect may be obtained by capsule materials that dissolve at different pH values, by capsules that release slowly by osmotic pressure, or by any other known means of controlled release. U.S. Pat. No. 6,306,406 to Deluca discloses a number of time-release methods and related references, the contents of which is incorporated herein.

In another preferred embodiment of treating existing tumors, the composition includes an effective amount to inhibit tumor growth of an adenosine A₃ receptor antagonist and a chemotherapeutic agent that is a taxane family compound, for example paclitaxel or docetaxel. Paclitaxel is commercially available under the tradename TAXOL from Bristol-Myers Squibb Company, Princeton, N.Y. and as a generic drug from IVAX Corp, Miami, Fla. Docetaxel is commercially available under the tradename TAXOTERE from Aventis Pharmaceuticals, Collegeville, Pa. In this embodiment the A₃ antagonist may reduce the growth rate of cancerous cells, interfere with adenosine protective effects against hypoxia, enhance the chemotherapeutic effect of the taxane, reduce hypersensitivity reactions in the patient and counter development of multi-drug resistance.

Materials and Methods

Chemicals and Reagents

Human A375, SKMES, HT29, Panc-1, Jurkat, HeL023 and NCTC2544 cells were obtained from American Tissue Culture Collection (ATCC). Tissue culture media and growth supplements were obtained from BioWhittaker. Unless otherwise noted, all other chemicals were purchased from Sigma. MRE 3046F20, 5-N-(4-methylphenyl-carbamoyl)amino-8-methyl-2 (2-furyl)-pyrazolo-[4,3e]-1,2,4-triazolo[1,5-c]pyrimidine; IL-10 salt, 5-N-(4-diethylamino-phenyl-carbamoyl)amino-8-methyl-(2(2furyl)-pyrazolo-[4,3e]1,2,4-triazolo[1,5-c]pyrimidine); MRE 3008F20, 5-N-(4-methoxyphenyl-carbamoyl)amino-8-propyl-2 (2furyl)-pyrazolo-[4,3e]1,2,4-triazolo[1,5-c]pyrimidine, MRE 3055F20, 5-N-(4-phenylcarbamoyl)amino-8-propyl-2 (2furyl)-pyrazolo[4,3e]-1,2,4triazolo-[1,5c]pyrimidine and MRE 3062F20, 5-N-(4-phenyl-carbamoyl)amino-8-butyl-2(2furyl)-pyrazolo-[4,3e]-1,2,4triazolo[1,5-c]pyrimidine were synthesized by Prof. P. G. Baraldi, University of Ferrara, Italy. CGS15943, 5-amino-9-chloro-2-(furyl) 1,2,4-triazolo[1,5-c]quinazoline and ZM 241385, 4-(2-[7-amino-2-(2-furyl)-[1,2,4]triazolo[2,3-a][1,3,5]triazin-5-ylamino]ethyl)phenol were obtained from RBI [Zeneca Pharmaceuticals, Cheshire, UK]. PD 98059, 2-Amino-3-methoxyflavone, (a selective inhibitor of MAP kinase (MEK)) and rhodamine 123 were obtained from Calbiochem. U0126 (an inhibitor of MEK-1 and MEK-2) and SB 203580 (an inhibitor of p38 MAP kinase) were from Promega. RNAse was purchased from Boehringer.

Cell Culture

For chemotherapeutic enhancement testing, the human melanoma A375, human lung carcinoma SKMES, colon carcinoma HT29, and pancreatic cancer Panc-1 cell lines were originally obtained from the ATCC (American Type Culture Collection) and have been maintained at PRC. Cell lines are maintained in RPMI-1640 medium supplemented with 100 units/ml penicillin G sodium, 100 μg/ml streptomycin sulfate, 0.25 μg/ml amphotericin B (fungizone), 2 mM glutamine, 10 mM HEPES, 25 μg/ml gentamycin, and 10% heat-inactivated fetal bovine serum. The cells are cultured in a T25 Falcon Tissue Culture flask in a humidified incubator at 37° C. with 5% CO₂-95% air. The cells are sub-cultured twice a week. The doubling times of A375, SKMES, HT29, and Panc-1 cultures are approximately 22, 46, 48, and 60 hr, respectively.

For multi-drug resistance testing, A375 and NCTC2544 cells are grown adherently and maintained in DMEM and EMEM medium, respectively, containing 10% fetal calf serum, penicillin (100 U/ml), streptomycin (100 μg/ml), L-glutamine (2 mM) at 37° C. in 5% CO₂/95% air. HeL023 and Jurkat were grown in RPMI-1640 medium, containing 10% fetal calf serum, penicillin (100 U/ml), streptomycin (100 μg/ml), L-glutamine (2 mM) at 37° C. in 5% CO₂/95% air. Cells are passaged two or three times weekly at a ratio between 1:5 and 1:10. Lymphocytes were isolated from buffy coats kindly provided by the Blood Bank of the University Hospital of Ferrara. Blood donated by healthy volunteers, after informed consent for research was obtained. Lymphocytes were isolated by density gradient centrifugation (Ficoll/Histopaque 1.077 g/ml). The cells are stimulated with purified phytohemoagglutinin (1 μg/ml) and expanded in RPMI medium added of interleukin 2 (20 Units/ml) and 10% fetal calf serum.

Colony Formation Assay

Exponentially growing A375 cells were seeded at 300 cells per well in six-well plates with 2 ml of fresh medium and treated with paclitaxel, vindesine and adenosine receptor agonists and antagonists dissolved in DMSO solution. Control plates received the same volume of DMSO alone. After 7 days of growth at 37° C. in humidified atmosphere containing 5% CO₂, the cells were fixed with absolute methanol for 5′ and stained with 1/10 Giemsa/phosphate-buffered saline (PBS) staining solution for 10 minutes. Staining solution was removed and colonies of greater than 30 cells were scored as survivors. For each treatment, six individual wells were scored.

Flow Cytometry Analysis

A375 and NCTC2544 adherent cells were trypsinized, mixed with floating cells, washed with PBS and permeabilised in 70% (vol/vol) ethanol/PBS solution at 4° C. for at least 24 hours. Jurkat, HeL023 and PBMC cells were centrifuged for 10 minutes at 1000×g. The cell pellet was then resuspended and permeabilised in 70% (vol/vol) ethanol/PBS solution at 4° C. for at least 24 hours. The cells were washed with PBS and the DNA was stained with a PBS solution, containing 20 μg/ml of propidium iodide and 100 μg/ml of RNAse, at room temperature for 30 minutes. Cells were analysed by FACScan (Becton-Dickinson) and the content of DNA was evaluated by the Cell-LISYS program (Becton-Dickinson). Cell distribution among cell cycle phases and apoptotic cells was evaluated as previously described (Secchiero et al., 2001). Briefly, the cell cycle distribution is shown as the percentage of cells containing 2n (G₁ phase), 4n (G₂ and M phases), 4n>x>2n. DNA amount (S phase) judged by propidium iodide staining. The apoptotic population is the percentage of cells with DNA content lower than 2n.

Determination of Chemotherapeutic Enhancement

The anti-proliferative activity of compounds can be readily determined using no more than routine experimentation using cell growth inhibitory assays. Selection of cell lines for such assays is based upon desired future pharmaceutical use. Numerous cell lines are available for study from American Type Culture Collection, Manassas, Va.

Cell lines suitable for assays include, but are not limited to, HL-60 human leukemia, A375 human melanoma, SKMES human lung carcinoma, HT29 human colon carcinoma and Panc-1 human pancreatic carcinoma. Cell lines can be maintained in RPMI-1640 medium supplemented with 100 units/ml penicillin G sodium, 100 μg/ml streptomycin sulfate, 0.25 μg/ml amphotericin B (fungizone), 2 mM glutamine, 10 mM HEPES, 25 μg/ml gentamycin, and 10% heat-inactivated fetal bovine serum. Such maintained cells can be cultured in a T25 Falcon Tissue Culture flask in a humidified incubator at 37° C. with 5% CO₂-95% air. For example, such maintained cells can be sub-cultured twice a week with approximate doubling times of 22 hours for A375 human melanoma, 46 hours for SKMES human lung carcinoma, 48 hours for HT29 human colon carcinoma and 60 hours for Panc-1 human pancreatic carcinoma.

One of the growth inhibitory assays is the MTT assay. Cells (1000-1500 cells/well) are seeded in a 96-well micro culture plate in a total volume of 100 μl/well. After overnight incubation in a humidified incubator at 37° C. with 5% CO₂-95% air, chemotherapeutic drug solutions diluted with culture medium at various concentrations are added in the amount of 100 μl to each well. The plates are placed in a humidified incubator at 37° C. with 5% CO₂-95% air for 7-10 days. The plates are then centrifuged briefly and 100 μl of the growth medium is removed. Cell cultures are incubated with 50 μl of MTT [3-(4,5-dimethylthiazol-2-yl)-2,5-diphenyl-tetrazolium bromide] reagent (1 mg/ml in Dulbecco's phosphate-buffered saline) for 4 hr at 37° C. The resultant purple formazan precipitate is solubilized with 200 μl of 0.04 N HCl in isopropanol. Absorbance is measured at a wavelength of 595 nm and at a reference wavelength of 655 nm using a Bio-Rad Model 3550 Microplate Reader. Preferably, all tests are run in duplicate for each dose level.

Bio-Rad brands Microplate Readers, when properly equipped, transmit measured test results to a personal computer for interpretation via computer programs such as the EZED50 program. The EZED50 computer program estimates the concentration of agent that inhibits cell growth by 50% as compared to the control cells. This is termed the IC₅₀ and is determined by curve fitting test data using the following four logistic equation.

$$Y = \frac{{A}_{max} - {A}_{min}}{1 + {( X / {IC}_{50} )}^{n}} + {A}_{min}$$

where Aₘₐₓ is the absorbance of the control cells, Aₘᵢₙ is the absorbance of the cells in the presence of the highest agent concentration, Y is the observed absorbance, X is the agent concentration, IC₅₀ is the concentration of agent that inhibits the cell growth by 50% compared to the control cells, and n is the slope of the curve.

If testing concentrations are properly selected (i.e. no or little growth inhibition at low concentrations and complete inhibition at high concentrations), EZED50 program fits the data extremely well and estimates the IC₅₀ value accurately. If the testing agent was too potent and inhibits cell growth by more than 50% at all of the concentrations tested, EZED50 cannot estimate the IC₅₀ value accurately (as indicated by an erroneously fitted Aₘₐₓ value). In these instances, the data could be re-analyzed by fixing the Aₘₐₓ value.

On the other hand, if the compound being tested produces incomplete inhibition at the highest agent concentration, EZED50 will overestimate the potency of the testing agent (the fitted IC₅₀ value is lower than the “actual” IC₅₀ value). In this case, the IC₅₀ could be estimated more accurately if both the Amax and Aₘᵢₙ values are fixed. Although it may be feasible to estimate IC₅₀ value by fixing Aₘₐₓ and/or Aₘₐₓ and Aₘᵢₙ without repeating the experiment, the best way to determine the IC₅₀ accurately is to decrease or increase the concentrations of the test agent and repeat the test.

Inhibitory assay testing is also used to determine enhanced therapeutic effects from combining A₃ receptor antagonists with other tumor inhibiting agents. The inhibitory cell growth assays are run with and without selected concentrations of A₃ receptor antagonists. An enhanced therapeutic effect is present when the IC₅₀ of the tumor inhibiting agent is lower with the A₃ receptor antagonist.

Quantitatively, the enhancement factor (EF) is calculated by dividing the IC₅₀ value of the tumor inhibiting agent for a tumor cell line by the IC₅₀ value of the agent with A₃ receptor antagonist for the same cell line:

$$Ehanced Factor ( EF ) = \frac{{IC}_{50} of anti \text{-} tumor agent}{{IC}_{50} of anti \text{-} tumor agent with {A}_{3} receptor antagonist}$$

Interpretation of the EF is dependent upon the concentrations of A₃ receptor antagonist tested. For example, when using very low concentrations of A₃ receptor antagonist, any EF above 1.0 is synergistic if the A₃ receptor antagonist concentration is below the threshold of cell growth inhibition. On the other hand, at high concentrations of A₃ receptor antagonist, any EF above 1.0 could be non-synergistic, and result primarily from the effects of the A₃ receptor antagonist. At concentrations of A₃ receptor antagonist near the IC₅₀ of the A₃ receptor antagonist, an EF above 2.0 is considered synergistic and an EF of 1.0 to 2.0 can be expected from geometric combination of the anti-tumor agent and the A₃ receptor antagonist.

Rhodamine 123 Efflux Assay

5×10⁵ cells from each subline were loaded with 50 ng/ml of rhodamine 123 for 30 minutes at 37° C. in medium. The cells were washed and resuspended in dye-staining agent free medium for 3 hours at 37° C. to allow rhodamine 123 efflux. The cells were then washed twice and the fluorescence of intracellular rhodamine 123 were analysed with Flow cytometer (residual fluorescence F_{RES}). The fluorescence was compared with loaded cells maintained at 4° C. to prevent drug export (maximal fluorescence F_{MAX}). The cells were treated with adenosine receptor antagonists to evaluate the ability of adenosine receptors to interfere with P-gp drug efflux activity. Cells from each subline that were not being exposed to rhodamine 123 were used as negative controls.

Metabolic Inhibitors

Cells were treated for 30 minutes with metabolic inhibitors or with drug vehicle (DMSO) prior to being challenged with adenosine receptor antagonists and paclitaxel or vindesine. After 24 hours, cells were collected for flow cytometry analysis. PD 98059 was used at 20 μM as an inhibitor of MEK to prevent MEK-1 activation. U01 26 was used at 10 μM as inhibitor of MEK-1 and MEK-2 to prevent extracellular signal-regulated kinase ERK-1 and ERK-2 activation. SB 203580 was used at 1 μM as an inhibitor of p38MAP kinase (p38^{MAPK}).

Statistical Analysis

All values in the figures and text are expressed as mean±standard deviation (SD) of n observation (with n>3). Data sets were examined by analysis of variance (ANOVA) and Dunnett's test (when required). A P value less than 0.05 was considered statistically significant. Representative images obtained by FACscan are reported, with similar results having been obtained in at least three different experiments.

### EXAMPLES

The following examples illustrate aspects of this invention but should not be construed as limitations. The symbols and conventions used in these examples are intended to be consistent with those used in the contemporary, international, chemical literature, for example, the Journal of the American Chemical Society (A J. Am. Chem. Soc.©) and Tetrahedron.

Three A₃ receptor antagonists have been tested for their Degree of Growth Inhibitory activity and enhancement of inhibitory growth function of known anti-neoplastic agents. Table 2 indicates receptor binding assay results for three A₃ receptor antagonist compounds. Structure of the three compounds is as follows: 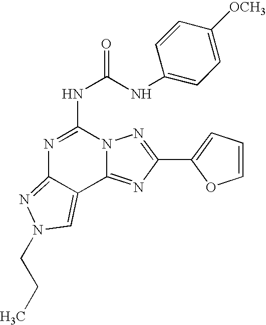
MRE3008F20: 5-[[(4-Methoxyphenyl)amino]carbonyl]amino-8-propyl-2-(2-furyl)-pyrazolo[4,3-e]1,2,4-triazolo[1,5-c]pyrimidine 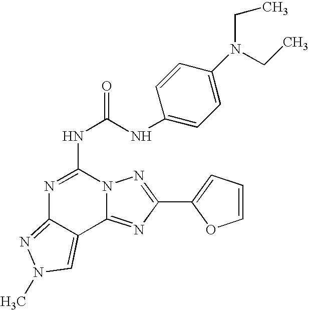
IL-10: N-1-(4-diethylamino-phenyl)-N′-5-[8-methyl-2-(2-furyl)-pyrazolo [4,3-e]1,2,4-triazolo[1,5-c]pyrimidine]-urea

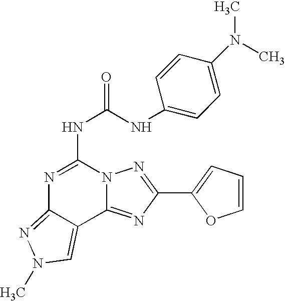

IL-11: N-1-(4-dimethylamino-phenyl)-N′-5-[8-methyl-2-(2-furyl)-pyrazolo[4,3-e]1,2,4-triazolo[1,5-c]pyrimidine]-urea

TABLE 2
Binding Affinity at rA₁, rA_{2A} and hA₃ Ad nosin Receptors

| | rA₁ (Kᵢ, | rA_{2A} | | | rA_{2A}/ |
|---|---|---|---|---|---|
| | nM) | (Kᵢ, nM) | hA₃ (Kᵢ, nM) | rA₁/hA₃ | hA₃ |
| MRE3008F20 | >10,000 | 1,993 | 0.29 | >34,482 | 6,872 |
| | | (1,658- | (0.27-0.32) | | |
| | | 2,397) | | | |
| IL-10 | 580 | 523 | 27 | 21.5 | 19.4 |
| IL-11 | >10,000 | >10,000 | 15 | >6700 | >6700 |

Table 2 shows MRE3008F20, IL-10 and IL-11 to be potent, selective antagonists for the human adenosine A₃ receptor.

In addition to binding affinity studies of these three compounds, growth inhibitory studies were also performed. Results of growth studies of the compounds, uncombined with other compounds, are shown in Table 3. Table 3 also indicates the growth inhibitory activity of common anti-neoplastic agents.

TABLE 3
Growth Inhibitory Activity of A₃ Antagonists and Anti-Neoplastic Agents Used Separately

| | IC₅₀ Values (ug/ml) ± Mean | | | |
|---|---|---|---|---|
| | Human | | Human | Human Lung |
| | Melanoma | Human Colon | Pancreatic Cancer | Carcinoma |
| Agent | A375 | Carcinoma HT29 | Panc-1 | SKMES |
| MRE3008F20 | 91.3 ± 1.5 | 39.4 ± 7.7 | 15.8 ± 13.5 | 19 ± 1.2 |
| IL-10 | 11.9 ± 0.4 | 10 ± 0.7 | 3.9 ± 3.3 | 5.9 ± 0.3 |
| IL-11 | 49.3 ± 40.7 | 56.5 ± 8.3 | 8 ± 5.7 | 36.3 ± 31.5 |
| irinotecan HCL | 0.88 ± 0.19 | 1.05 ± 0.07 | 1.12 ± 0.29 | 1.23 ± 0.36 |
| paclitaxel | 0.004 ± 0.0002 | 0.00112 ± 0.0003 | 0.0027 ± 0.0003 | 0.0026 ± 0.0007 |
| docetaxel | 0.0002 ± 0.00007 | 0.00024 ± 0.00005 | 0.00037 ± 0.00031 | 0.00084 ± 0.00037 |
| vinblastine | 0.0016 | 0.0019 ± 0.0004 | 0.0018 ± 0.0008 | 0.0019 ± 0.0009 |

Compounds MRE3008F20, IL-10 and IL-11 were obtained from King Pharmaceutical, Inc. Irinotecan HCL (Camptosar®; 20 mg/ml) was obtained from Pharmacia & Upjohn Co. Paclitaxel (Taxol®, 6 mg/ml) was obtained from Bristol-Myers Squibb, Co. Docetaxel (Taxotere®) was obtained from Rhone-Poulenc Rorer. Vinblastine sulfate salt was obtained from Sigma Chemical Co., (V1377). Irinotecan was diluted with culture medium. All other agents were dissolved in 100% DMSO at appropriate concentrations. The DMSO stock solutions were diluted 100-fold with growth medium so that the final DMSO concentration was 1%. We have previously shown that DMSO has no effect on the growth of culture cells at concentrations up to 1%. MTT (3-[4,5-Dimethylthiazol-2-yl]2,5-diphenyltetrazolium bromide) was obtained from Sigma Chemical Co. RPMI-1640 medium, antibiotic antimycotic 100 X consisting of 10,000 units/ml penicillin G sodium, 10,000 μg/ml streptomycin sulfate, 25 μg/ml amphotericin B (fungizone), glutamine (200 mM), HEPES buffer (1 M), gentamicin (50 mg/ml), sodium bicarbonate (7.5%), and fetal bovine serum were obtained from GibcoBRL. The complement in fetal bovine serum was inactivated at 56° C. for 30 min.

The human melanoma A375, human lung carcinoma SKMES, colon carcinoma HT29, and pancreatic cancer Panc-1 cell lines were originally obtained from the ATCC (American Type Culture Collection) and have been maintained in RPMI-1640 medium supplemented with 100 units/ml penicillin G sodium, 100 μg/ml streptomycin sulfate, 0.25 μg/ml amphotericin B (fungizone), 2 mM glutamine, 10 mM HEPES, 25 μg/ml gentamycin, and 10% heat-inactivated fetal bovine serum.

Most of the agents showed a broad spectrum and equally potent growth inhibitory activity against these four histologically distinct human tumor cell lines (A375 melanoma, HT29 colon carcinoma, Panc-1 pancreatic carcinoma, and SKMES lung carcinoma). The IC₅₀ values were approximately the same against all four cell lines. Interestingly, HT29 is approximately 30-fold more refractory than the A375 cell line to doxorubicin (IC₅₀ value of 0.211 vs. 0.0064 μg/ml) and mitoxantrone (IC₅₀ value of 0.1 vs. 0.0032 μg/ml), respectively. It is possible that the HT29 cell line has an altered form of topoisomerase II, and is therefore more refractory to doxorubicin and mitoxantrone, which mediate their growth inhibitory activity by trapping topoisomerase II, DNA, and drug in ternary complexes.

### Example 1

### Human Melanoma A375

The growth inhibitory activity of paclitaxel against the human melanoma A375 cell line was determined in the absence or in the presence of 10 μg/ml of MRE3008F20 or 5 μg/ml each of IL-10 and IL-11 (Table 4). At these sub-cytotoxic concentrations, MRE3008F20, IL-10, and IL-11 (approximately 30-45% growth inhibition in the presence of A₃ antagonists alone), enhanced the growth inhibitory activity of paclitaxel by 8-12-fold; IC₅₀ values decreased from 0.0046 μg/ml (paclitaxel alone) to 0.0004-0.00054 μg/ml (paclitaxel plus MRE3008F20, IL-10, and IL-11).

TABLE 4
Growth Inhibitory Activity of A₃ Antagonists and
Anti-Neoplastic Agents Used Jointly With A375 Cells

| Anti-Neoplastic Agent | A₃ Antagonist | IC₅₀ | Enhancement |
|---|---|---|---|
| (Concentration Range) | (Concentration) | (μg/ml) | Factor |
| paclitaxel (0.0002-0.1 μg/ml) | none | 0.0046 | |
| paclitaxel (0.0002-0.1 μg/ml) | MRE3008F20 (10 μg/ml) | 0.00054 | 8.5 |
| paclitaxel (0.0002-0.1 μg/ml) | IL-10 (5 μg/ml) | 0.0005 | 9.2 |
| paclitaxel (0.0002-0.1 μg/ml) | IL-11 (5 μg/ml) | 0.0004 | 11.5 |

It was also determined that IL-10 and IL-11 enhance the growth inhibitory activity of paclitaxel in a concentration-dependent manner. Results of further testing of the enhancement effects with taxane family compounds is given in Table 5. Both of the common taxane compounds paclitaxel (taxol™) and docetaxel (taxotere™) are included.

Table 5 illustrates concentration-dependencies for concentrations of 1, 3, and 10 μg/ml for MRE3008F20 and for concentrations of 0.5, 1.5, and 5 μg/ml for compounds IL-10 and IL-11. MRE3008F20 enhanced the growth inhibitory activity of paclitaxel in a concentration-independent manner (enhancement factor=6.4-7.1 at all three concentrations). On the other hand, IL-10 and IL-11 enhanced the growth inhibitory activity of paclitaxel in a concentration-dependent manner. This testing also indicated that compounds MRE3008F20, IL-10 and IL-11 have enhancement factors with docetaxel that are indicative of a synergistic effect.

TABLE 5
Growth Inhibitory Activity of A₃ Antagonists and
Taxane Compounds used Jointly with A375 Cells

| Anti-Neoplastic Agent | A3 Antagonist | IC50 | Enhancement |
|---|---|---|---|
| (Concentration Range) | (Concentration) | (μg/ml) | Factor |
| paclitaxel (0.0001-0.05 μg/ml) | none | 0.0042 | |
| paclitaxel (0.0001-0.05 μg/ml) | MRE3008F20 (1 μg/ml) | 0.00059 | 7.1 |
| paclitaxel (0.0001-0.05 μg/ml) | MRE3008F20 (3 μg/ml) | 0.00066 | 6.4 |
| paclitaxel (0.0001-0.05 μg/ml) | MRE3008F20 (10 μg/ml) | 0.00059 | 7.1 |
| paclitaxel (0.0001-0.05 μg/ml) | none | 0.0044 | |
| paclitaxel (0.0001-0.05 μg/ml) | IL-10 (0.5 μg/ml) | 0.0012 | 3.7 |
| paclitaxel (0.0001-0.05 μg/ml) | IL-10 (1.5 μg/ml) | 0.00061 | 7.2 |
| paclitaxel (0.0001-0.05 μg/ml) | IL-10 (5 μg/ml) | 0.0005 | 8.8 |
| paclitaxel (0.0001-0.05 μg/ml) | none | 0.0044 | |
| paclitaxel (0.0001-0.05 μg/ml) | IL-11 (0.5 μg/ml) | 0.0014 | 3.1 |
| paclitaxel (0.0001-0.05 μg/mi) | IL-11 (1.5 μg/ml) | 0.00067 | 6.6 |
| paclitaxel (0.0001-0.05 μg/ml) | IL-11 (5 μg/ml) | 0.00046 | 9.6 |
| docetaxel(0.00001-0.005 μg/ml) | none | 0.000038 | |
| docetaxel(0.00001-0.005 μg/ml) | MRE3008F20 (1 μg/ml) | 0.0000006 | 63.3 |
| docetaxel(0.00001-0.005 μg/ml) | MRE3008F20 (3 μg/ml) | 0.0000055 | 6.9 |
| docetaxel(0.00001-0.005 μg/ml) | MRE3008F20 (10 μg/ml) | 0.0000072 | 5.3 |
| docetaxel(0.00001-0.005 μg/ml) | none | 0.000021 | |
| docetaxel(0.00001-0.005 μg/ml) | IL-10 (0.5 μg/ml) | 0.0000025 | 8.4 |
| docetaxel(0.00001-0.005 μg/ml) | IL-10 (1.5 μg/ml) | 0.0000023 | 9.1 |
| docetaxel(0.00001-0.005 μg/ml) | IL-10 (5 μg/ml) | 0.0000072 | 2.9 |
| docetaxel(0.00001-0.005 μg/ml) | none | 0.000046 | |
| docetaxel(0.00001-0.005 μg/ml) | IL-11 (0.5 μg/ml) | 0.0000087 | 5.3 |
| docetaxel(0.00001-0.005 μg/ml) | IL-11 (1.5 μg/ml) | 0.000005 | 9.2 |
| docetaxel(0.00001-0.005 μg/1) | IL-11 (5 μg/ml) | 0.0000071 | 6.5 |

### Example 2

### Human Lung Carcinoma SKMES

In human lung carcinoma SKMES, all three A₃ antagonists enhance the growth inhibitory activity of paclitaxel, docetaxel, irinotecan and vindesine (Table 6).

TABLE 6
Growth Inhibitory Activity of A₃ Antagonists and Anti-Neoplastic
Agents used Jointly with SKMES Cells

| Anti-Neoplastic Agent | A₃ Antagonist | IC₅₀ | Enhancement |
|---|---|---|---|
| (Concentration Range) | (Concentration) | (μg/ml) | Factor |
| paclitaxel (0.0001-0.05 μg/ml) | none | 0.0033 | |
| paclitaxel (0.0001-0.05 μg/ml) | MRE3008F20 (1 μg/ml) | 0.0008 | 4.1 |
| paclitaxel (0.0001-0.05 μg/ml) | IL-10 (0.5 μg/ml) | 0.0012 | 2.8 |
| paclitaxel (0.0001-0.05 μg/ml) | IL-11 (0.5 μg/ml) | 0.00078 | 4.2 |
| docetaxel(0.00001-0.005 μg/ml) | none | 0.00047 | |
| docetaxel(0.00001-0.005 μg/ml) | MRE3008F20 (1 μg/ml) | 0.00013 | 3.6 |
| docetaxel(0.00001-0.005 μg/ml) | IL-10 (0.5 μg/ml) | 0.00017 | 2.8 |
| docetaxel(0.00001-0.005 μg/ml) | IL-11 (0.5 μg/ml) | 0.00025 | 1.9 |
| irinotecan HCL (0.04-20 μg/ml) | none | 1.17 | |
| irinotecan HCL (0.04-20 μg/ml) | MRE3008F20 (1 μg/ml) | 0.57 | 2.1 |
| irinotecan HCL (0.04-20 μg/ml) | IL-10 (0.5 μg/ml) | 0.67 | 1.7 |
| irinotecan HCL (0.04-20 μg/ml) | IL-11 (0.5 μg/ml) | .061 | 1.9 |
| vinblastine(0.00001-0.005 μg/ml) | none | 0.0015 | |
| vinblastine(0.00001-0.005 μg/ml) | MRE3008F20 (1 μg/ml) | 0.0013 | 1.2 |
| vinblastine(0.00001-0.005 μg/ml) | IL-10 (0.5 μg/ml) | 0.0011 | 1.4 |
| vinblastine(0.00001-0.005 μg/ml) | IL-11 (0.5 μg/ml) | 0.00094 | 1.6 |

In comparing Table 6 with the results obtained with A375 human melanoma, the magnitude of enhancement for the taxane compounds in SKMES cells is less than that observed in A375 melanoma. These differences in absolute magnitude of synergism may be related to the activity of P-gp in the different tumors under the test conditions employed. However, the enhancement between A₃ antagonists and taxane compounds is consistently observed. Enhancement in SKMES cells is also consistent with a mechanism of inhibiting P-gp, in the case of vinblastine, and inhibiting MRP, as in the case of irinotecan HCL.

### Example 3

### Human Colon Carcinoma HT29

The growth inhibitory activity of paclitaxel and docetaxel against the human colon carcinoma HT29 cell line was determined in the absence or in the presence of 1 μg/ml of MRE3008F20 or 0.5 μg/ml each of IL-10 and IL-11 (Table 7). It was found that A₃ antagonists enhance the growth inhibitory activity of paclitaxel, docetaxel., doxorubicin and irinotecan.

TABLE 7
Growth Inhibitory Activity of A₃ Antagonists and
Taxane Compounds Used Jointly With HT29 Cells

| Anti-Neoplastic | A₃ Antagonist | IC₅₀ | Enhancement |
|---|---|---|---|
| (Concentration Range)Agent | (Concentration) | (μg/ml) | Factor |
| paclitaxel (0.0001-0.05 μg/ml) | none | 0.0025 | |
| paclitaxel (0.0001-0.05 μg/ml) | MRE3008F20 (1 μg/ml) | 0.00085 | 2.9 |
| paclitaxel (0.0001-0.05 μg/ml) | IL-10 (0.5 μg/ml) | 0.00100 | 2.5 |
| paclitaxel (0.0001-0.05 μg/ml) | IL-11 (0.5 μg/ml) | 0.00099 | 2.5 |
| docetaxel(0.00001-0.005 μg/ml) | none | 0.000018 | |
| docetaxel(0.00001-0.005 μg/ml) | MRE3008F20 (1 μg/ml) | 0.0000012 | 15.0 |
| docetaxel(0.00001-0.005 μg/ml) | IL-10 (0.5 μg/ml) | 0.0000033 | 5.5 |
| docetaxel(0.00001-0.005 μg/ml) | IL-11 (0.5 μg/ml) | 0.0000028 | 6.4 |
| doxorubicin (0.0002-0.1 μg/ml) | none | 0.46 | |
| doxorubicin (0.0002-0.1 μg/ml) | MRE3008F20 (1 μg/ml) | 0.28 | 1.6 |
| doxorubicin (0.0002-0.1 μg/ml) | IL-10 (0.5 μg/ml) | 0.31 | 1.5 |
| doxorubicin (0.0002-0.1 μg/ml) | IL-11 (0.5 μg/ml) | 0.38 | 1.2 |
| irinotecan HCl (0.04-20 μg/ml) | none | 0.96 | |
| irinotecan HCl (0.04-20 μg/ml) | MRE3008F20 (1 μg/ml) | 0.53 | 1.8 |
| irinotecan HCl (0.04-20 μg/ml) | IL-10 (0.5 μg/ml) | 0.77 | 1.2 |
| irinotecan HCI (0.04-20 μg/ml) | IL-11 (0.5 μg/ml) | 0.85 | 1.1 |

### Example 4

### Human Pancreatic Cancer Panc-1

In human pancreatic cancer Panc-1, A₃ antagonists potentiated the growth inhibitory activity of taxane family compounds paclitaxel and docetaxel (Table 8). However, the potentiation observed is of a smaller magnitude compared to that observed in A375 melanoma.

TABLE 8
Growth Inhibitory Activity of A₃ Antagonists and
Taxane Compounds Used Jointly With Panc-1 Cells

| Anti-Neopiastic Agent | A₃ Antagonist | IC₅₀ | Enhancement |
|---|---|---|---|
| (Concentration Range) | (Concentration) | (μg/ml) | Factor |
| paclitaxel (0.0001-0.05 μg/ml) | none | 0.0029 | |
| paclitaxel (0.0001-0.05 μg/ml) | MRE3008F20 (1 μg/ml) | 0.0015 | 1.9 |
| paclitaxel (0.0001-0.05 μg/ml) | IL-10 (0.5 μg/ml) | 0.0017 | 1.7 |
| paclitaxel (0.0001-0.05 μg/ml) | IL-11 (0.5 μg/ml) | 0.0016 | 1.8 |
| docetaxel(0.00001-0.005 μg/ml) | none | 0.00068 | |
| docetaxel(0.00001-0.005 μg/ml) | MRE3008F20 (1 μg/ml) | 0.00038 | 1.8 |
| docetaxel(0.00001-0.005 μg/ml) | IL-10 (0.5 μg/ml) | 0.00047 | 1.4 |
| docetaxel(0.00001-0.005 μg/ml) | IL-11 (0.5 μg/ml) | 0.00050 | 1.4 |

It is seen from the above data and tables that the use of high affinity adenosine A3 receptor antagonists in conjunction with chemotherapeutic cancer agents result in a notable enhancement effect for many of the agents.

### Examples

### MDR Cancer Cell Treatments

Having noted that chemotherapeutic cancer agents can be selected for A₃ antagonist enhancement from the form of MDR they are associated with, the inventors have further established that high affinity A₃ adenosine receptor antagonists can be used to counter P-gp and MRP associated multi-drug resistance.

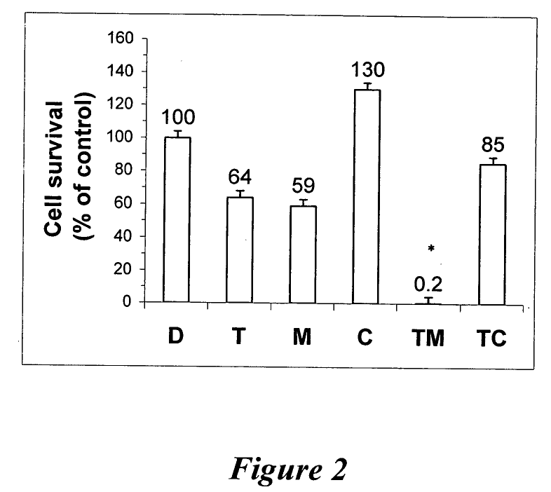

At first, inventors evaluated whether A₃ adenosine receptor could protect the cell by the toxic effect of conventional chemotherapeutic drug. Colony formation assay experiments were performed on A375 melanoma cells treated with increasing concentration of paclitaxel (0.25-75 ng/ml). A₃ stimulation is achieved with the selective agonist CI-IB-MECA while A₃ blockade is obtained with the selective antagonist MRE 3008F20. Colony formation of A375 cells is abolished when both paclitaxel (0.75 ng/ml) and MRE 3008F20 (10 μM) are applied whereas colony formation is only partly decreased when paclitaxel alone or MRE 3008F20 alone are applied. (FIG. 2)

Colony formation of A375 cells is increased of about 30% when the adenosine A₃ agonist CI-IB-MECA (10 μM) is applied. This is seen in the “C” bar of FIG. 2 being 130% of the DMSO control bar “D.” FIG. 2 further shows that increased colony formation occurs when CI-IB-MECA is combined with the taxane family compound paclitaxel (0.75 ng/ml). When paclitaxel alone is added (“T” bar of FIG. 2), colony formation is 64% of the control. Colony formation is increased 32% to 85% of control when the CI-IB-MECA is combined (“TC” bar of FIG. 2).

Using the adenosine A₃ antagonist MRE 3008F20 (10 μM) alone decreases colony formation to 59% of control (“M” bar of FIG. 2). Surprisingly, when the A₃ antagonist is combined with the taxane compound, virtually all colony formation ceases (“TM” bar of FIG. 2). This clearly identifies the synergistic nature of combining A₃ antagonists with chemotherapeutic agents. In comparison to the surprising result of 0.2% colony formation, a geometrical combination predicts a result of 38%.

One explanation for these results is that A₃ receptors trigger a pro-survival signal, not able to restore colony formation ability of A375 cells treated with the taxane paclitaxel, while the blockage of A₃ increased paclitaxel mediated deleterious effects (p<0.01, “TM” bar versus “T” bar of FIG. 2).

Subsequent to colony formation experiments, the inventors also analysed the ability of A₃ adenosine receptor antagonists to enhance chemotherapeutic effects by performing an acute treatment of A375 cells with the taxane family compound paclitaxel and the vinca alkaloid vindesine. Cell proliferation and apoptosis are quantified by flow cytometer analysis after propidium-iodide (PI) DNA staining. At 24 hours post-exposure, paclitaxel and vindesine induced a dose dependent cell accumulation into G₂/M cell cycle phases and a parallel decrease of the G₁ population.

To quantify the effect of paclitaxel and vindesine to alter cell proliferation the inventors determined the concentration exerting the 50% of the G₂/M accumulation (EC₅₀). When exposed to increasing concentrations of chemotherapeutic drugs, EC₅₀ are 16.60±2.00 ng/ml and 1.90±0.20 nM for paclitaxel and vindesine, respectively (means of four experiments). Analyses further show that EC₅₀ of paclitaxel and vindesine for decrease of G₁ population are not significantly changed respect to EC₅₀ of G₂/M arrest. The S-phase population, representative of replicating DNA, is not appreciably changed.

Additional experiments quantified the sub-G, population, representative of cells undergoing apoptosis. In A375 cells, the percentage of apoptotic cells increases progressively with paclitaxel concentration, reaching the maximum value (ranging from 35 to 53% on different experiments) at 5 ng/ml. An increase of the paclitaxel concentration results in a decrease of A375 cells at sub-G₁. The concentration exerting the maximal apoptosis (EC_{MAX}) is 6.00±0.63 ng/ml (mean of four experiments). Similarly, experiments performed with vindesine obtain an EC_{MAX} value of 3.54±0.42 nM (mean of four experiments).

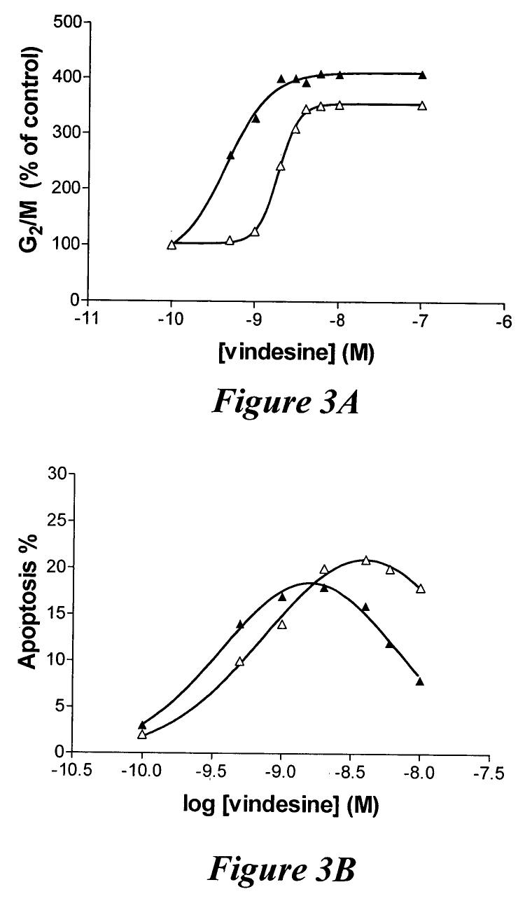

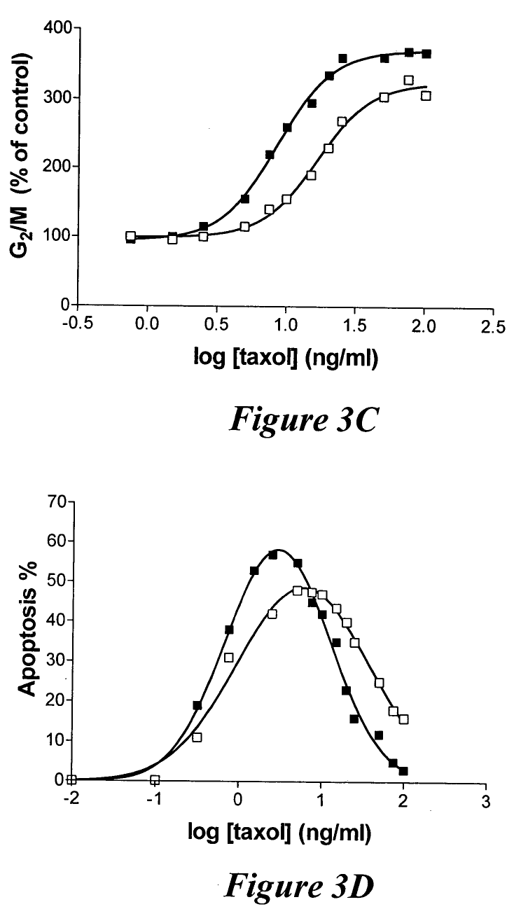

A375 cells treated with paclitaxel or with vindesine with or without the A₃ adenosine receptor selective A₃ antagonist MRE 3008F20 demonstrate enhancement. FIGS. 3A and 3C show that MRE 3008F20 (10 μM) improved vindesine and paclitaxel ability to alter cell proliferation: MRE 3008F20 reduced paclitaxel and vindesine EC₅₀ of 1.9 and 4.0 fold, respectively. Similar results were obtained analysing EC₅₀ values calculated for the G₁ population.

Furthermore, MRE 3008F20 (10 μM) reduced EC_{MAx} of 2.0 and 2.1 fold, for paclitaxel and vindesine, respectively (FIGS. 3B and 3D).

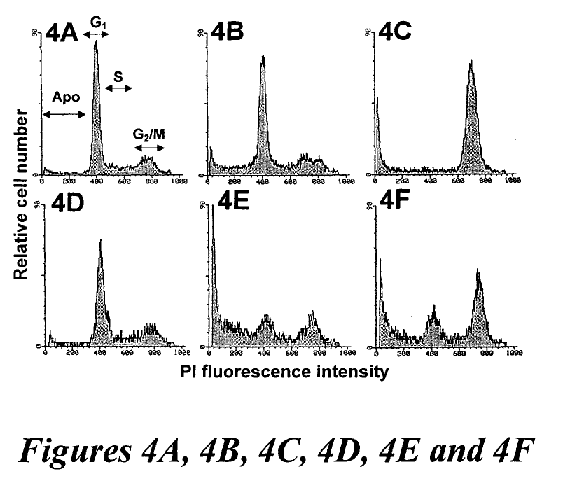

Representative flow cytometry profiles of DNA content in A375 cells (Apo (sub diploid cells), G₁, S and G₂/M phases) are shown in FIG. 4. FIG. 4C shows that, under both treatments (A₃ antagonist MRE3008F20 plus vinca alkaloid vindesine), vindesine response increased as cells progressed from G₁ to G₂/M phases respect to vindesine-treated cells alone (FIG. 4B). Similar results were obtained with the taxane compound paclitaxel (FIG. 4E-F).

To verify whether this enhancement activity was not related to toxic contaminants present in the MRE3008F20 solution but due to A₃ adenosine receptor specific blockade, inventors quantified the ability of other adenosine receptor antagonists to enhance A375 cell responses to vindesine and paclitaxel.

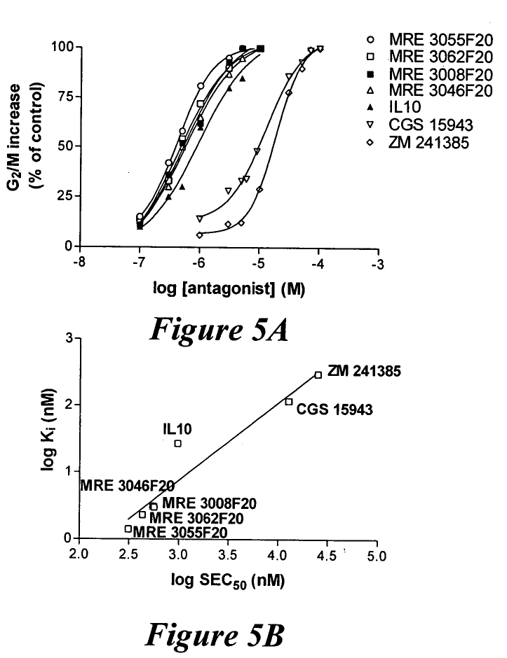

A375 cells were treated with vindesine (1 nM) with increasing concentrations of adenosine receptor antagonists (MRE3055F20, MRE3062F20, MRE3046F20, MRE3008F20, IL-10, CGS 15943, ZM 241385). As illustrated in FIG. 5A, the order of potency of adenosine antagonists to enhance vindesine effect was: MRE3055F20 (highest)>MRE3062F20>MRE 3046F20>MRE3008F20>IL-10>CGS15943>ZM241385 (lowest). The concentrations exerting the 50% of the enhancing activity (SEC₅₀) are reported in Table 9 as the mean of four experiments. SEC₅₀ values are in good agreement with inhibitory equilibrium binding constants (Ki) observed in binding experiments for the adenosine A₃ receptor (FIG. 5B). Table 9 Adenosine receptor antagonist parameters of enhancing activity to vindesine and binding affinity to A₃ adenosine receptor in A375 cells.

| | SEC₅₀ | Kᵢ |
|---|---|---|
| | (μM) | (nM) |
| MRE 3055F20 | 0.31 ± 0.03 | 1.4 ± 0.2 |
| MRE 3062F20 | 0.43 ± 0.04 | 2.3 ± 0.2 |
| MRE 3046F20 | 0.57 ± 0.06 | 3.0 ± 0.3 |
| MRE 3008F20 | 0.55 ± 0.06 | 3.1 ± 0.3 |
| IL-10 | 0.97 ± 0.10 | 30.0 ± 2.5 |
| CGS 15943 | 12.60 ± 1.41 | 118 ± 12 |
| ZM 241385 | 25.00 ± 2.23 | 270 ± 25 |

SEC₅₀: adenosine receptor antagonist dose that induces 50% of the enhancing activity to vindesine (1 nM), calculated on G₂/M accumulation dose response curve. Ki: equilibrium constant of binding affinity at human A₃ adenosine receptor. Data represents the mean of four independent experiments.

To verify the role of A₃ receptor antagonists on paclitaxel and vindesine mediated alteration of cell proliferation and apoptosis, testing was performed with additional cell lines. Cell lines were selected with a pattern of surface adenosine receptor expression similar to A375 cell line, namely HeL023, Jurkat, NCTC2544 and PBMC blasts. Experimental conditions are similar to those for the A375 cell testing. The effect upon EC₅₀ values of vindesine and paclitaxel mediated cell cycle distribution alteration (G₂/M accumulation) under treatment with the A3 antagonist MRE 3008F20 is given in Table 10. The variation in the level of apoptosis as reflected in EC_{MAX} values are also indicated.

In Table 10, the HeL023 cell line has the lowest sensitivity to paclitaxel and vindesine alone. Interestingly, HeL023 cells also show the greatest amount of enhancement by MRE3008F20 co-treatment.

TABLE 10
Induction of G₂/M accumulation and apoptosis of different cells by paclitaxel
and vindesine with or without A₃ receptor antagonist MRE3008F20 treatment

| | A375 | | HeL023 | | Jurkat | |
|---|---|---|---|---|---|---|
| | paclitaxel | vindesine | paclitaxel | vindesine | paclitaxel | vindesine |
| | EC₅₀ | EC₅₀ | EC₅₀ | EC₅₀ | EC₅₀ | EC₅₀ |
| DMSO | 16.60 ± 2.00 | 1.90 ± 0.20 | 50.60 ± 5.30 | 25.80 ± 3.25 | 2.96 ± 0.31 | 0.93 ± 0.08 |
| MRE 3008F20 | 8.60 ± 0.81* | 0.48 ± 0.05* | 4.10 ± 0.40* | 0.71 ± 0.08* | 2.52 ± 0.30* | 0.37 ± 0.04* |
| | EC_{MAX} | EC_{MAX} | EC_{MAX} | EC_{MAX} | EC_{MAX} | EC_{MAX} |
| DMSO | 6.00 ± 0.63 | 3.54 ± 0.42 | 33.42 ± 3.55 | 33.11 ± 4.02 | 3.32 ± 0.52 | nd |
| MRE 3008F20 | 3.04 ± 0.32* | 1.66 ± 0.20* | 3.19 ± 0.30* | 1.05 ± 0.09* | 2.29 ± 0.31* | nd |

| | |
|---|---|
| NCTC 2544 | PBMC |

| | paclitaxel | vindesine | paclitaxel | vindesine |
|---|---|---|---|---|
| | EC₅₀ | EC₅₀ | EC₅₀ | EC₅₀ |
| DMSO | 2.69 ± 0.28 | 0.89 ± 0.08 | 5.30 ± 0.60 | 1.10 ± 0.90 |
| MRE 3008F20 | 2.41 ± 0.30 | 0.51 ± 0.06* | 5.30 ± 0.60 | 0.86 ± 0.09 |
| | EC_{MAX} | EC_{MAX} | EC_{MAX} | EC_{MAX} |
| DMSO | 2.69 ± 0.23 | 2.50 ± 0.27 | nd | nd |
| MRE 3008F20 | 2.55 ± 0.24 | 1.34 ± 0.15* | nd | nd |

Data represent the mean ± SE of four independent experiments. MRE3008F20 is 10 μM.
*P<0.01 vs DMSO; analysis was by ANOVA followed by Dunnett's test.

EC₅₀ values were obtained by analysing G₂/M accumulation dose response curve. EC_{MAX}values were obtained by analysing sub-G₁ accumulation dose response curve. Paclitaxel values have units of ng/ml. Vindesine values have units of nM. nd: not done.

The greater relative enhancement in the HeL023 cell line may be related to a factor absent in other cell lines or related to the intracellular concentration of actives that is modulated by P-glycoprotein drug expulsion activity.

Rhodamine 123 (Rh 123) retention, a P-gp functional assay, was studied in all cell lines. This assay was performed by incubating cells with Rh 123 and determining Rh 123 accumulation by measuring its fluorescence. The maximum load of Rh 123 (F_{MAX}) was quantified at the end of treatment harvesting the cells and storing them at 4° C. to prevent any active Rh123 efflux. The P-gp drug efflux was allowed incubating Rh 123-loaded cells in fresh new medium Rh 123-free for 3 hours at 37° C. After this incubation, residual fluorescence (FRES) was measured and compared to F_{MAx}.

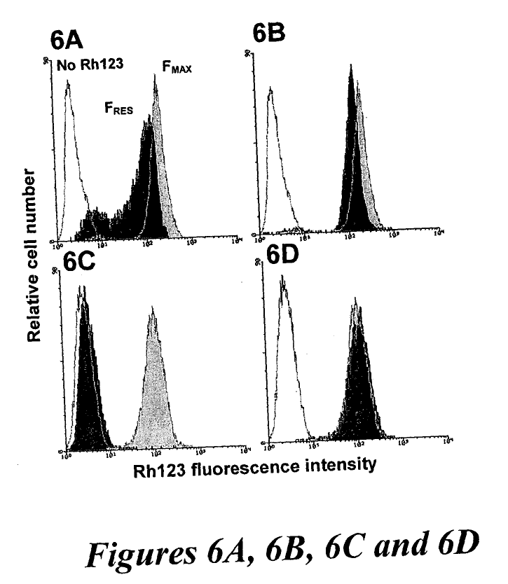

Results of the Rh123 retention in A375 cells is shown in FIG. 6A and FIG. 6B. FIG. 6A shows the flow chromatogram for Rh123 accumulation by A375 cells when the adenosine A3 antagonist is not present. Two cell populations are found, characterised by an FRES having a mean fluorescence intensity lower than F_{MAx}. The population with the lowest fluorescence, accounting for 26±5% of total cells, represents the cells expressing functional P-gp and having low intracellular level of Rh 123.

In contrast, FIG. 6B shows the A375 cellular accumulation of Rh123 in the presence of MRE3008F20 (10 μM). With the high affinity A₃ antagonist present, the response yielded a FRES chromatogram comparable to F_{MAx}, consistent with a completely blockade of P-gp mediated Rh123 transport.

Results of the Rh123 retention in human herithro-leukemia HeL023 cells is shown in FIG. 6C and FIG. 6D. FIG. 6C shows that HeL023 cells have higher P-gp activity than A375 cells. FRES (dark shaded area) is similar to the chromatogram obtained with cells not treated with Rh123 (autofluorescence). This is consistent with nearly all (100±1%) the cells expressing high level of functional P-gp.

With the addition of the A₃ antagonist MRE3008F20, HEL023 cells showed a P-gp inhibitor behaviour (FIG. 6D). FRES in the presence of MRE 3008F20, is similar to F_{MAX}. This is indicative of near total P-gp inhibition.

The Jurkat and NCTC2544 cells studied did not appear to have a significant change of Rh123 fluorescence after 3 hours of incubation at 37° C. (F_{RES} was similar to F_{MAX}) consistent with the absence or not detectable P-pg drug pumping activity in these cells.

To verify that the anti-P-gp activity of A₃ antagonists is not related to toxic contaminants present in the MRE3008F20 solution, the ability of other adenosine receptor antagonists to interfere with P-gp mediated drug-efflux was quantified. HeL023 cells were treated with adenosine receptor antagonists (IL-10, MRE3008F20, MRE3055F20, MRE3062F20, MRE3046F20, CGS15943 and ZM241385). Results are given in Table 11.

Table 11 shows the percentage of cells expressing P-gp activity (% of Rh 123 negative cells) in the presence of adenosine receptor antagonists. The high affinity adenosine A₃ antagonists IL-10, MRE3008F20, MRE3055F20, MRE3062F20 and MRE3046F20 are strong inhibitors of P-gp activity. In contrast the low affinity antagonists, ZM241385 and CGS15943 had much lower effect on P-gp activity. This may be due to the higher A₃ affinity or due to the existence of a structure-activity relationship for the inhibition of P-gp drug expulsion activity mediated by adenosine receptor antagonists.

TABLE 11
Rhodamine 123 efflux (Rh123) evaluation in HeL023 cells in the presence and
absence of adenosine receptor antagonists.

| Treatment | Concentration (μM) | % of Rh123 negative cells |
|---|---|---|
| DMSO | | 99.7 ± 1.2 |
| IL10 | 10 | 0.4 ± 0.2 |
| 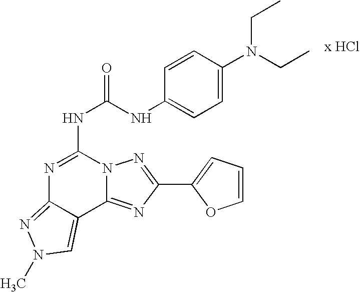 | | |
| MRE 3008F20 | 10 | 0.5 ± 0.3 |
| 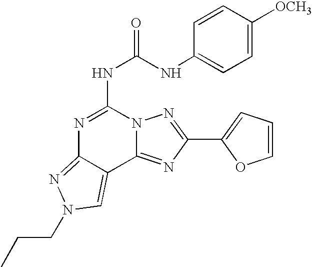 | | |
| MRE 3055F20 | 10 | 0.8 ± 0.2 |
| 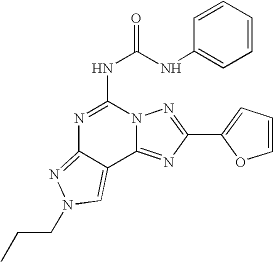 | | |
| MRE 3062F20 | 10 | 1.0 ± 0.2 |
| 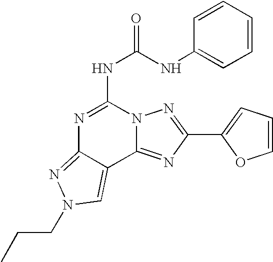 | | |
| MRE 3046F20 | 10 | 22.0 ± 1.0 |
| 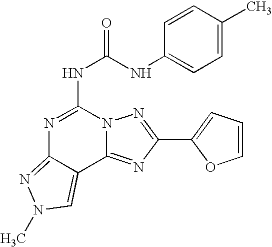 | | |
| CGS 15943 | 70 | 87.1 ± 1.0 |
| 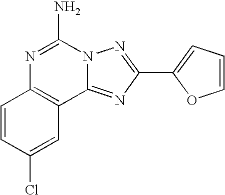 | | |
| ZM 241385 | 70 | 93.0 ± 1.1 |
| 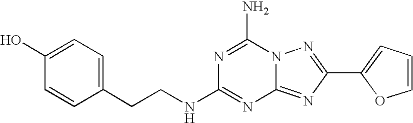 | | |

Table 11 data represents the mean of four independent experiments.

To underline the molecular mechanisms sustaining the A₃ antagonist mediated response to vindesine and paclitaxel anti-proliferative effects, signalling studies were performed.

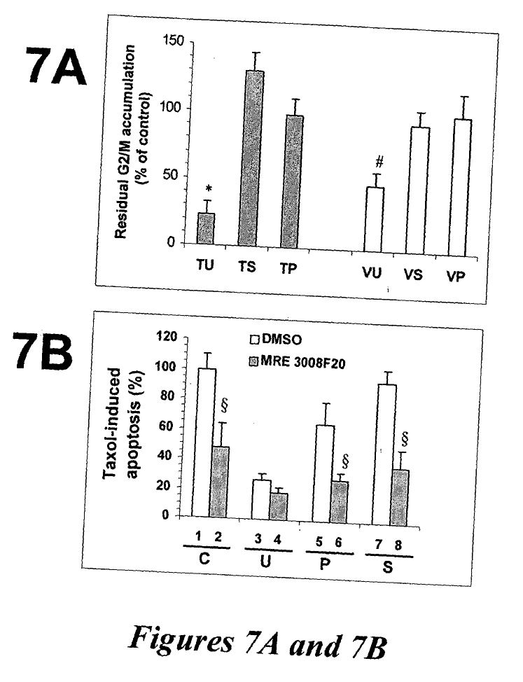

Previous research has related paclitaxel and Vinca alkaloid derivatives lethality to activation of mitogen-activated protein kinase (MAPK) family (Lieu in Sequence-dependent potentiation mol. Pharmacol. 2001). Three MAPK family members have been characterised thus far: ERKs (or p42/44^{MAPK}) JNK (or SAPK) and p38^{MAPK}. Signalling studies investigated the effect of ERKs and p38^{MAPK} on MRE 3008F20 mediated enhancement to vindesine and to paclitaxel. The JNK pathway was not studied due to the lack of commercially available JNK inhibitor. Results are shown in FIG. 7A and FIG. 7B.

PD98059, a selective inhibitor of MEK1/2 (dudley dt, PNAS 92:7686, 1995), was used to inhibit the MEK pathways. U0126, an agent approximately 100-fold more potent than PD98059, was used as inhibitor of ERK activation. SB203580 selectively inhibits p38^{MAPK} activity (Young PR JBC 272:12116 1997). A375 cells were pretreated with PD98059 (20 μM), U0126 (30 μM), SB203580 (1 μM) or with DMSO (as control) and then challenged with vindesine (1 nM) or with paclitaxel (15 ng/ml). At 24 hours post treatment, cells were harvested and apoptosis and cell cycle were analysed.

The combination of PD98059, U0126 and SB203580 plus paclitaxel or vindesine did not alter the G₂/M accumulation rate respect to paclitaxel or vindesine alone. PD98059 and SB203580 failed to inhibit MRE3008F20 (10 μM) improved susceptibility of A375 cells to vindesine and paclitaxel. Results for PD98059 with paclitaxel is shown as bar “TP” of FIG. 7A and with vindesine is bar “VP”. The results for SB203580 plus paclitaxel is shown as bar “TS” of FIG. 7A and with vindesine is bar “VS”.

U0126 prevented MRE3008F20-induced accumulation in G₂/M by 23±5% and by 48±5% in presence of paclitaxel (bar “TU” of FIG. 7A) and vindesine (bar “VU” of FIG. 7A), respectively. This infers that the molecular mediator of enhancement activity was ERK.

The effects on selectively blocking the pathways is shown in FIG. 7B. Noted is a significant reduction of apoptosis induced by paclitaxel 25 ng/ml in cells treated with PD98059 (35±6%, FIG. 7B, lane 5) and U0126 (73±10%, FIG. 7B, lane 3), whereas SB203580 did not exert any effect (FIG. 7B, lane 7). However, PD98059 and SB203580 (FIG. 7B, lanes 6 and 8, respectively) failed to enhance the protective effect of MRE3008F20 in apoptosis induced by paclitaxel, as observed in presence of U0126 (FIG. 7B, lane 4).

These results provide confirmation that the MRE3008F20-induced reduction of apoptosis of paclitaxel is dependent on ERK activation. Additional testing demonstrated similar trends in HeL023, NCTC2544 and Jurkat cell lines (data not shown).

Further testing indicates that P-gp interference and ERK engagement can occur independently of each other. A375 cells were first treated with U0126 (30 μM) for 30 minutes and subsequently incubated with MRE3008F20 (10 μM). The P-gp activity is compared which those of cells treated with MRE3008F20 alone (for example, cells of FIG. 6B). U0126 failed to prevent MRE 3008F20 blockade of P-gp. This confirms that P-gp interference and ERK engagement can occur independently of each other.

Discussion

Results presented show that several adenosine receptor antagonists exert enhancing activity to chemotherapeutic agents. Noteworthy, this enhancing activity is A₃ adenosine receptor dependent. This pharmacological specificity was determined by an accurate Spermean's rank correlation between the dose exerting physiological effect (SEC50 quantified on G₂/M accumulation) and the binding ability to A₃ adenosine receptors (Ki) of different adenosine receptor antagonists (r=0.96, FIG. 5B). A known and clear explanation is not yet available as to the mechanism of this enhancement. However, the high statistically significant Spermean's rank correlation coefficient between SEC50 values in the G₂/M accumulation and receptor affinity values showed a highly significant positive correlation.

Results show that A₃ adenosine receptor antagonists enhance chemotherapeutic agent anti-proliferative and apoptotic effects. The A₃ adenosine receptor antagonists reduce EC₅₀ doses of chemotherapeutic drugs (quantified by analysing G₂/M accumulation rate) to 12.3, 1.9, 1.2-fold for paclitaxel and 36.3, 4.0, 2.5-fold for vindesine when challenged on HeL023, A375 and Jurkat cell lines, respectively. This enhancement activity was confirmed also when the apoptotic degree was evaluated: EC_{MAX} dose of chemotherapeutic drugs are reduced to 10.5, 2.0, 1.5-fold for paclitaxel on HeL023, A375 and Jurkat cells, respectively, and 31.5, 2.0-fold for vindesine on HeL023 and A375 cells, respectively.

The variable degree of EC₅₀ (and EC_{MAX}) value observed in different cell types suggest a cell type specific participation in drug-induced enhancement. Of note, the prerequisite of the drug activity is its delivery to the target site. However, efficiency of drug is limited by appearing resistance, i.e. lack of cell sensitivity to the administered drug. The tumor cells with multidrug resistance (MDR) phenotype are characterised by lowered intracellular accumulation of the compounds they are resistant to.

Moreover, most cell lines with MDR phenotype show the over-expression of 170 KDa membrane associated P-glycoprotein (P-gp) that acts as an energy-dependent efflux pump. It has been demonstrated that this protein plays an important role in the transport of toxic endogenous metabolites and it seems to be responsible for the decreased intracellular drug accumulation observed in resistant cells (Fojo A, Cancer Res 45:3002-7, 1985). Previous studies reported that melanoma and HeL heamopoietic cell line expressed functional P-gp.

On the other hand, MRP and not P-gp transporter is expressed in Jurkat leukemia cells (T lymphocytic cell line). This is consistent with the results as HeL023 and A375 cells produced a P-gp efflux-activity whereas in Jurkat cells had low rhodamine 123 efflux. It is shown that adenosine A₃ antagonists are useful for enhancement in both P-gp expressing and MRP expressing cell lines.

The much lower ability of CGS15943 and ZM241385 to inhibit P-pg and MRP correlates with the lower adenosine A₃ affinity of these compounds. The high affinity A₃ antagonist compounds tested have greater potency in both enhancement and inhibiting P-gp and MRP drug resistance. This may be due to the higher affinity or to the molecular structure of such compounds. For example, the tested high affinity compounds have a phenyl-carbamoyl-amino derivative in the N5 position of a 2-furylpyrazolo-triazolo-pyrimidine structure. CGS15943 and ZM241385 in addition to having much lower A₃ affinity, also have very different molecular structures.

### Example

### Medicaments Comprising Adenosine A₃ Receptor Antagonists

The amount of a compound required to be effective as an antagonist of adenosine A₃ receptors will, of course, vary with the active moiety selected, the individual mammal being treated and is ultimately at the discretion of the medical or veterinary practitioner. The factors to be considered include the binding affinity of the active, the route of administration, the nature of the formulation, the mammal's body weight, surface area, age and general condition, and the particular compound to be administered. However, a suitable effective dose is in the range of about 0.1 pg/kg to about 10 mg/kg body weight per day, preferably in the range of about 1 mg/kg to about 3 mg/kg per day.

The total daily dose may be given as a single dose, multiple doses, e.g., two to six times per day, or by intravenous infusion for a selected duration. Dosages above or below the range cited above are within the scope of the present invention and may be administered to the individual patient if desired and necessary. For example, for a 75 kg mammal, a dose range would be about 75 mg to about 220 mg per day, and a typical dose would be about 150 mg per day. If discrete multiple doses are indicated, treatment might typically be 50 mg of a compound of the present invention given 3 times per day.

Formulations

Formulations of the present invention for medical use comprise an active compound, i.e., a high affinity adenosine A₃ receptor antagonist, together with an acceptable carrier thereof and optionally other therapeutically active ingredients. The carrier must be pharmaceutically acceptable in the sense of being compatible with the other ingredients of the formulation and not deleterious to the recipient thereof.

The present invention, therefore, further provides a pharmaceutical formulation comprising a high affinity adenosine A₃ receptor antagonist together with a pharmaceutically acceptable carrier thereof.

The formulations include, but are not limited to, those suitable for oral, rectal, topical or parenteral (including subcutaneous, intramuscular and intravenous) administration. Preferred are those suitable for oral or parenteral administration.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there are a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch.

Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients) may include flavoring, an agent to retard crystallization of the sugar or an agent to increase the solubility of any other ingredient, e.g., as a polyhydric alcohol, for example, glycerol or sorbitol.

The high affinity adenosine A₃ receptor antagonist, alone or in combination with other suitable components, can be made into aerosol formulations to be administered via inhalation. These aerosol formulations can be placed into pressurized acceptable propellants, such as dichlorodifluoromethane, propane, nitrogen,. and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The high affinity adenosine A₃ receptor antagonist can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical excipients and adjuvants.

Topical formulations for high affinity adenosine A₃ receptor antagonists include ointments, creams, gels and lotions that may be prepared by conventional methods known in the art of pharmacy. In addition to the ointment, cream get, or lotion base and the A₃ antagonists, such topical formulation may also contain preservatives, perfumes, and additional active pharmaceutical agents. Preferred additional pharmaceutical agents include the chemotherapeutic agents for cancer treatments noted to be enhanced or benefited by the A₃ antagonist (for example by preventing MDR). An example of a preferred topical formulation includes a high affinity adenosine A₃ receptor antagonist and a taxane family compound.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The high affinity adenosine A₃ receptor antagonists may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the high affinity adenosine A₃ receptor antagonist may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Formulations for rectal administration may be presented as a suppository with a conventional carrier, e.g., cocoa butter or Witepsol S55 (trademark of Dynamite Nobel Chemical, Germany), for a suppository base.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

In addition to the aforementioned ingredients, the formulations of this invention may further include one or more cytotoxic agent as well as one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

### Example Formulations

The following examples illustrate aspects of this invention but should not be construed as limitations. The symbols and conventions used in these examples are indented to be consistent with those used in the contemporary, international, chemical literature, for example, the Journal of the American Chemical Society and Tetrahedron.

### Example

### Pharmaceutical Formulations

(A) Transdermal System - for 1000 patches

| Ingredients | Amount | |
|---|---|---|
| Active compound | 100 | g |
| Silicone fluid | 450 | g |
| Colloidal silicon dioxide | 2 | g |

The silicone fluid and active compound are mixed together and the colloidal silicone dioxide is added to increase viscosity. The material is then dosed into a subsequent heat sealed polymeric laminate comprised of the following: polyester release liner, skin contact adhesive composed of silicone or acrylic polymers, a control membrane which is a polyolefin, and an impermeable backing membrane made of a polyester multilaminate. The resulting laminated sheet is than cut into 10 sq. cm patches

(B) Oral Tablet - For 1000 Tablets

| Ingredients | Amount | |
|---|---|---|
| Active compound | 50 | g |
| Starch | 50 | g |
| Magnesium Stearate | 5 | g |

The active compound and the starch are granulated with water and dried. Magnesium stearate is added to the dried granules and the mixture is thoroughly blended. The blended mixture is compressed into tablets.

(C) Injection - for 1000, 1 mL Ampules

| Ingredients | Amount | |
|---|---|---|
| Active compound | 10 | g |
| Buffering Agents | q.s. | |
| Propylene glycol | 400 | mg |
| Water for injection | q.s. 1000 | mL |

The active compound and buffering agents are dissolved in the propylene glycol at about 50° C. The water for injection is then added with stirring and the resulting solution is filtered, filled into ampules, sealed and sterilized by autoclaving.

(D) Continuous Injection - for 1000 mL

| Ingredients | Amount |
|---|---|
| Active compound | log |
| Buffering agents | q.s. |
| Water for injection | q.s. 1000 mL |

The active compound and buffering agents are dissolved in water at about 50° C. The resulting solution is filtered, filled into appropriate administration container, sealed and sterilized.

(E) Topical Ointment - for 1000, 1 g packs

| Ingredients | Amount | |
|---|---|---|
| Active compound | 10 | g |
| White petrolatum base | q.s. 990 | g |

The active compound is blended into the petrolatum base under sterile conditions and filled into 1 gram packs.

Although the present invention has been described in terms of specific embodiments, various substitutions of materials and conditions can be made as will be known to those skilled in the art. For example, other excipients may be utilized in preparing the pharmaceutical formulations. In addition, many of the active adenosine A₃ receptor antagonists contain one or more asymmetric centers and may therefore give rise to enantiomers and diastereomers as well as their racemic and resolved, enantiomerically pure or diastereomerically pure forms, and pharmaceutically acceptable salts thereof. It is often desirable that the adenosine A₃ receptor antagonists be given simultaneously with the cytotoxic agent. When this is the case, users of this invention may find it advantageous to combine the A₃ receptor antagonists with the cytotoxin into a single dosage form. These and other variations will be apparent to those skilled in the art and are meant to be included herein. The scope of the invention is only to be limited by the following claims:

1. A method of synergistically enhancing the chemotherapeutic treatment of cancer expressing adenosine A₃ receptors comprising administering to a mammal in need thereof an effective amount of a high affinity adenosine A₃ receptor antagonists either prior to or during administration of a chemotherapeutic cancer agent.

2. The method of claim 1 wherein the chemotherapeutic cancer agent is a taxane family compound.

3. The method of claim 1 wherein the chemotherapeutic cancer agent is a vinca alkaloid compound.

4. The method of claim 1 wherein the chemotherapeutic cancer agent is a camptothecin compound.

5. The method of claim 1 wherein the chemotherapeutic cancer agent is an antibiotic compound.

6. The method of claim 1 wherein the high affinity adenosine A₃ receptor antagonist is a compound of formula: 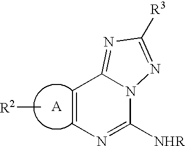
wherein:
A is imidazole, pyrazole, or triazole;
R is —C(X)R¹, —C(X)—N(R¹)₂, —C(X)OR¹, —C(X)SR¹, —SOₙR¹, —SOₙSR¹, or —SOₙ—N(R¹)₂;
R¹ is hydrogen, alkyl, substituted alkyl, alkenyl, substituted alkenyl, alkynyl, substituted alkynyl, aryl, substituted aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms;
R² is hydrogen, alkyl, substituted alkyl, alkenyl, aralkyl, substituted aralkyl, heteroaryl, substituted heteroaryl or aryl;
R³ is furan, pyrrole, thiophene, benzofuran, benzypyrrole, benzothiophene, optionally substituted with one or more substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aryl, substituted aryl, aryloxy, azido, carboxyl, carboxylalkyl, cyano, halo, nitro, heteroaryl, heteroaryloxy, heterocyclic, heterocyclooxy, aminoacyloxy, thioalkoxy, substituted thioalkoxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl;
X is O, S, or NR¹; and
pharmaceutically acceptable salts thereof.

7. The method of claim 1 wherein the high affinity adenosine A₃ receptor antagonist is a compound of formula: 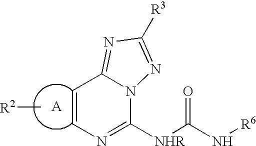
wherein:
A is imidazole, pyrazole, or triazole;
R² is hydrogen, alkyl, substituted alkyl, alkenyl, aralkyl, substituted aralkyl, heteroaryl, substituted heteroaryl or aryl; R³ is furan; and
R⁶ is aryl, substituted aryl, heteroaryl, substituted heteroaryl, heterocycle or substituted heterocycle; and
pharmaceutically acceptable salts thereof.

8. The method of claim 6 wherein R² is selected from the group consisting of hydrogen, alky, alkenyl and aryl.

9. The method of claim 6 wherein A is a triazolo ring.

10. The method of claim 6 wherein A is a pyrazolo ring.

11. The method of claim 1 wherein the cancer is selected from the group consisting of human leukemia, melanoma, pancreatic carcinoma, breast carcinoma, prostrate carcinoma, colon carcinoma, ovarian carcinoma, lung carcinoma, histiocytic lymphoma, astrocytoma and keratinocytoma.

12. A method of synergistically enhancing the chemotherapeutic treatment of cancer expressing adenosine A₃ receptors comprising administering to a mammal in need thereof an effective amount of a high affinity adenosine A₃ receptor antagonists either prior to or during administration of a chemotherapeutic cancer agent wherein the cancer has multi-drug resistance that is P-glycoprotein dependent.

13. The method of claim 11 wherein the chemotherapeutic cancer agent is a taxane family compound.

14. The method of claim 11 wherein the chemotherapeutic cancer agent is a vinca alkaloid compound.

15. The method of claim 11 wherein the chemotherapeutic cancer agent is a camptothecin compound.

16. The method of claim 11 wherein the chemotherapeutic cancer agent is an antibiotic compound.

17. The method of claim 11 wherein the high affinity adenosine A₃ receptor antagonist is a compound of formula: 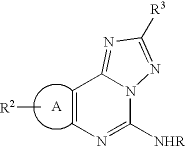
wherein:
A is imidazole, pyrazole, or triazole;
R is —C(X)R¹, —C(X)—N(R¹)₂, —C(X)OR¹, —C(X)SR¹, —SOₙR¹, —SOₙSR¹, or —SOₙ—N(R¹)₂;
R¹ is hydrogen, alkyl, substituted alkyl, alkenyl, substituted alkenyl, alkynyl, substituted alkynyl, aryl, substituted aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms;
R² is hydrogen, alkyl, substituted alkyl, alkenyl, aralkyl, substituted aralkyl, heteroaryl, substituted heteroaryl or aryl;
R³ is furan, pyrrole, thiophene, benzofuran, benzypyrrole, benzothiophene, optionally substituted with one or more substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aryl, substituted aryl, aryloxy, azido, carboxyl, carboxylalkyl, cyano, halo, nitro, heteroaryl, heteroaryloxy, heterocyclic, heterocyclooxy, aminoacyloxy, thioalkoxy, substituted thioalkoxy, —SO-alkyl, —SO-substituted alkyl, —SO-aryl, —SO-heteroaryl, —SO2-alkyl, —SO2-substituted alkyl, —SO2-aryl, —SO2-heteroaryl, and trihalomethyl;
X is O, S, or NR¹; and
pharmaceutically acceptable salts thereof.

18. The method of claim 11 wherein the high affinity adenosine A₃ receptor antagonist is a compound of formula: 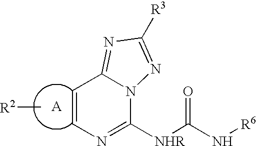
wherein:
A is imidazole, pyrazole, or triazole;
R² is hydrogen, alkyl, substituted alkyl, alkenyl, aralkyl, substituted aralkyl, heteroaryl, substituted heteroaryl or aryl; R³ is furan; and
R⁶ is heteroaryl or substituted heteroaryl; and
pharmaceutically acceptable salts thereof.

19. The method of claim 16 wherein R2 is selected from the group consisting of hydrogen, alkyl, alkenyl and aryl.

20. The method of claim 16 wherein A is a triazolo ring.

21. The method of claim 16 wherein A is a pyrazolo ring.

22. The method of claim 11 wherein the cancer is selected from the group consisting of human leukemia, melanoma, pancreatic carcinoma, breast carcinoma, prostrate carcinoma, colon carcinoma, ovarian carcinoma, lung carcinoma, histiocytic lymphoma, astrocytoma and keratinocytoma.

23. A method of synergistically enhancing the chemotherapeutic treatment of cancer expressing adenosine A₃ receptors comprising administering to a mammal in need thereof an effective amount of a high affinity adenosine A₃ receptor antagonist and a adenosine-5′-triphosphate depleting agent either prior to or during administration of a chemotherapeutic cancer agent wherein the cancer has multi-drug resistance that is P-glycoprotein dependent.

24. The method of claim 21 wherein the adenosine-5′-triphosphate depleting agent consists of a compound selected from the group consisting of L-alanosine and adenosine kinase inhibitors.

25. The method of claim 21 wherein the adenosine-5′-triphosphate depleting agent consists of a compound or a salt of a compound selected from the group consisting of 2-deoxyglucose, cyanine, oligomycin, valinomycin, and azide.

26. A method of treating skin carcinoma comprising administering to a human patient in need thereof an effective amount of a high affinity adenosine A₃ receptor antagonist either prior to or during administration of a chemotherapeutic cancer agent wherein the chemotherapeutic cancer agent is a taxane family compound.

27. The method of claim 24 wherein the high affinity adenosine A₃ receptor antagonist is administered in a topical application.
